(19) United States
(12) Patent Application Publication
(10) Pub. No.: US 2023/0017590 A1
(43) Pub. Date: Jan. 19, 2023

(54) COMPOSITIONS AND METHODS FOR CULTURING HEMATOPOIETIC STEM AND PROGENITOR CELLS

(71) Applicant: Deverra Therapeutics Inc., Seattle, WA (US)

(72) Inventors: Tao Bai, Lexington, MA (US); Devikha Chandrasekaran, Seattle, WA (US); Mary Prieve, Lake Forest Park, WA (US); Colleen Delaney, Seattle, WA (US)

(73) Assignee: Deverra Therapeutics Inc., Seattle, WA (US)

(21) Appl. No.: 17/780,370

(22) PCT Filed: Nov. 27, 2020
(86) PCT No.: PCT/US2020/062507

§ 371 Date: May 26, 2022

Related U.S. Application Data
(60) Provisional application No. 62/941,563, filed on Nov. 27, 2019.

(51) Int. Cl. C12N 5/00; C12N 5/0789

(52) U.S. Cl. CPC C12N 5/0068; C12N 5/0087; C12N 5/0647; C12N 2533/32; C12N 2500/34

(57) ABSTRACT

The present disclosure provides compositions and methods for culturing hematopoietic stem and progenitor cells, while maintaining or increasing the subpopulation of hematopoietic stem cells (HSCs). The methods and compositions describe us a 3D zwitterionic hydrogel to provide a biocompatible culture microenvironment for culturing encapsulated hematopoietic stem and progenitor cells.

### CROSS-REFERENCE TO RELATED APPLICATION

This application claims the benefit of U.S. Provisional Application No. 62/941,563, filed Nov. 27, 2019, the disclosure of which is incorporated herein in its entirety.

### BACKGROUND

Hematopoietic stem and progenitor cells (HSPCs) have many uses in medicine, both for cell therapy and as precursors for generation of other therapeutic cell types, such as T cells, NK cells and macrophages. Cord blood transplantation of hematopoietic stem and progenitor cells, CD34⁺ HSPCs in particular, is an application of such cells. Recently, administration of expanded populations of hematopoietic stem and progenitor cells has been studied as a means to bridge cord blood transplantation and as a means for reducing infection associated with pre-transplantation conditioning of patients.

HSPCs are also a source of other cell types within the hematopoietic lineage, such as T cells, NK cells, and macrophages. To prepare these cell types, HSPCs are typically cultured ex vivo to expand and differentiate them into the desired cell type. Ex vivo expansion of HSPCs can be performed in static or dynamic systems, typically using culture plates, flasks, bags, and the like, or bioreactors. Such systems can involve culturing HSPCs in 2-dimensional systems (2D), such as, for example, tissue culture plates in which the HSPCs generally form monolayers of suspension, or in 3-dimensional (3D) systems, in which HSPCs are cultured on or within a matrix.

While 2D and 3D systems can be effective to generate certain cell types, one of the challenges with 2D and 3D systems is maintaining hematopoietic stem cells (HSCs), a subpopulation of cells within the HSPCs. HSCs retain the potential to differentiate into other cell types but are typically quiescent. Typically, during culture in the 2D or 3D system the percentage of HSCs decreases over time. The HSCs tend to transform into multipotent progenitor cells (MPPs) and further to hematopoietic progenitor cells (e.g., myeloid or lymphoid progenitors) or more committed cell types (lineage⁺) over time. As such, these systems are not suitable for culturing and maintaining highly enriched populations of HSPCs, in which the percentage of HSCs in the cell culture is maintained or increased. Thus, there remains a need for compositions and methods for culturing HSPCs in which the percentage of HSCs is maintained or increased.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides compositions and methods for culturing hematopoietic stem and progenitor cells (HSPCs), while maintaining or increasing the subpopulation of HSCs. In some embodiments, the methods and compositions described herein use an alginate poly-lysine 3D zwitterionic hydrogel to provide a biocompatible culture microenvironment for culturing encapsulated HSPCs, while maintaining the subpopulation of HSCs or HSCs and MPPs. Advantageously, neither the encapsulation nor the depolymerization/recovery process significantly reduced cell viability. Furthermore, high HSPC viability can be maintained during the encapsulation and culture of the HSPCs, such that greater than 75% viability is maintained over the entire culturing step. This result indicates the methods of culturing HSPCs encapsulated within the alginate-poly-lysine 3D hydrogel described herein can be used for the culture and maintenance of HSPCs, and maintenance and/or expansion of HSCs and MPPs within the cell population of HSPCs.

In some embodiments, a method is provided for culturing hematopoietic stem and progenitor cells (HSPCs), comprising: providing at least one HSPC population comprising hematopoietic stems cells (HSCs; Lin⁻ CD34⁺ CD38⁻ CD133⁺ CD45RA⁻ CD90⁺); enriching the HSPC population for CD34⁺ or CD133⁺ HSPCs to prepare an enriched HSPC population that has been depleted of T cells and red blood cells; encapsulating the enriched HSPCs in a hydrogel comprising alginate and poly-lysine to form encapsulated HSPCs; and culturing the encapsulated HSPCs in a culture medium comprising interleukin-3 (IL-3), interleukin-6 (IL-6), thrombopoietin (TPO), Flt3-Ligand (Flt3-L), and stem cell factor (SCF), for a sufficient time to produce an expanded HSPC population, wherein the percentage of HSCs in the expanded HSPC population is the same as or greater than the percentage of HSCs in the enriched HSPC population.

In some embodiments, a method is provided for culturing hematopoietic stem and progenitor cells (HSPCs), comprising: providing at least one enriched CD34⁺ or CD133⁺ HSPC population comprising hematopoietic stems cells (HSCs; Lin⁻ CD34⁺ CD38⁻ CD133⁺ CD45RA⁻ CD90⁺), less than 2% T cells and less than 2% red blood cells; encapsulating the enriched HSPCs in a zwitterionic hydrogel comprising alginate and poly-lysine to form encapsulated HSPCs; culturing the encapsulated HSPCs in a culture medium comprising interleukin-3 (IL-3), interleukin-6 (IL-6), thrombopoietin (TPO), Flt3-Ligand (Flt3-L), and stem cell factor (SCF), for a sufficient time to produce an expanded HSPC population, wherein the percentage of HSCs in the hydrogel expanded HSPC population is the same or greater than the percentage of HSCs in the enriched HSPCs.

In some embodiments, the percentage of multi-potent progenitor cells (MPPs; Lin⁻ CD34⁺ CD38⁻ CD133⁺ CD45RA⁻ CD90⁻) in the hydrogel expanded HSPC population is the same as or greater than the percentage of MPPs in the enriched HSPCs.

In some embodiments, the enriched HSPC population is derived from umbilical cord blood, placental blood, or somatic stem cells. In some embodiments, the enriched HSPC population is derived from somatic stem cells of the peripheral blood mononuclear cells (PBMCs). In some embodiments, the enriched HSPC population is derived from umbilical cord blood or placental blood. In some embodiments, the enriched HSPC population is derived from at least two different sources of umbilical cord blood and/or placental blood that have not been immunologically matched to each other, or to a recipient. In some embodiments, the enriched HSPCs are not derived from somatic cells, embryonic stem cells, or induced pluripotent stem cells.

In some embodiments, the culture medium does not comprise a serum supplement or a serum supplement replacement. In some embodiments, the culture medium does not comprise fetal bovine serum, human serum albumin and/or human platelet lysate. In some embodiments, the culture medium does not comprise exogenous interleukin 15 (IL-15), interleukin 7 (IL-7), interleukin 2 (IL-2), Granulocyte-Colony Stimulating Factor (G-CSF), Granulocyte Macrophage-Colony Stimulating Factor (GM-CSF), Leukemia Inhibitory Factor (LIF), or Macrophage Inflammatory Protein-1 alpha (MIP-1a). In some embodiments, the culture medium does not comprise an aryl hydrocarbon receptor antagonist. In some embodiments, the culture medium and hydrogel do not comprise fibronectin, and/or fragments thereof. In some embodiments, the culture medium and hydrogel do not comprise exogenous feeder cells. In some embodiments, the culture medium and hydrogel do not comprise a Notch ligand. In some embodiments, a Notch ligand is attached to the hydrogel. In some embodiments, the poly-lysine is poly-L-lysine.

In some embodiments, the percentage of HSCs in the hydrogel expanded HSPC population is at least two times greater than the percentage of HSCs in the enriched HSPC population. In some embodiments, the percentage of HSCs in the hydrogel expanded HSPC population at the end of the culturing step is at least two times greater than the percentage of HSCs in the encapsulated HSPC population at the beginning of the culturing step. In some embodiments, the percentage of HSCs and MPPs in the hydrogel expanded HSPC population at the end of the culturing step is at least two times greater than the percentage of HSCs and MPPS in the encapsulated HSPC population at the beginning of the culturing step.

In some embodiments, the number of HSCs in the hydrogel expanded HSPC population is at least five times, at least ten times, at least 20 times, at least 40 times, at least 50 times, or more, greater than the number of HSCs in the encapsulated enriched HSPC population. In some embodiments, the number of HSCs in the hydrogel expanded population at the end of the culturing step is at least five times, at least ten times, at least 20 times, at least 40 times, at least 50 times, or greater than the number of HSCs in the encapsulated HSPC population at the beginning of the culturing step. In some embodiments, the number of HSCs and MPPs in the hydrogel expanded HSPC population at the end of the culturing step is at least ten times greater than the percentage of HSCs and MPPs in the encapsulated HSPC population at the beginning of the culturing step.

In some embodiments, the enriched HSPC population has been genetically modified. In some embodiments, the encapsulated HSPC population is genetically modified during culturing. In some embodiments, the hydrogel expanded HSPC population has been genetically modified to introduce a wild type version of a gene into the genome of at least some of the HSPCs. In some embodiments, the encapsulated HSPC population is cultured for about 2 to about 21 days. In some embodiments, the encapsulated HSPC population is cultured for about 7 to about 15 days.

In some embodiments, the enriched HSPCs comprise about 25% to about 95%, 50% to about 95% HSPCs or about 75% to about 95% HSPCs. In some embodiments, at least some of the HSCs in the expanded HSCs are quiescent. In some embodiments, the hydrogel expanded HSPC population comprises at least about 5%, at least about 10%, or at least about 15% HSCs, or at least about 5%, at least about 10% or at least about 15% HSCs and MPPs. In some embodiments, the hydrogel expanded HSPCs are released from the hydrogel.

In some embodiments, the hydrogel expanded HSPCs differentiate into hematopoietic progenitor cells (myeloid or lymphoid; HPCs), T cells, NK cells, CD20⁺ B cells, CD14⁺ monocytes, CD15⁺ neutrophils, and/or macrophages.

In some embodiments, a composition of enriched hematopoietic stem and progenitor cell (HSPC) population is provided, the composition comprising: at least about 25% or at least about 50% HSPCs comprising hematopoietic stems cells (HSCs; Lin⁻ CD34⁺ CD38⁻ CD133⁺ CD45RA⁻ CD90⁺), less than 2% T cells and less than 2% red blood cells that are encapsulated in a zwitterionic hydrogel comprising alginate and poly-lysine. The composition can further comprise a culture medium comprising interleukin-3 (IL-3), interleukin-6 (IL-6), thrombopoietin (TPO), Flt3-Ligand (Flt3-L), and stem cell factor (SCF). In some embodiments, HSPCs are derived from umbilical cord blood, placental blood or somatic stem cells. In some embodiments, the HSPCs are from somatic stem cells in the PBMCs. In some embodiments, the HSPCs are derived from umbilical cord blood or placental blood. In some embodiments, the HSPCs are derived from at least two different sources of umbilical cord blood and/or placental blood that have not been immunologically matched to each other, or a recipient. In some embodiments, the HSPCs are not derived from somatic cells, embryonic stem cells or induced pluripotent stem cells.

In some embodiments, the culture medium does not comprise a serum supplement or a serum supplement replacement. In some embodiments, the culture medium does not comprise fetal bovine serum, human serum albumin, or human platelet lysate. In some embodiments, the culture medium does not comprise exogenous IL-15, IL-7, IL-2, G-CSF, GM-CSF, LIF, or MIP-1a. In some embodiments, the culture medium does not comprise an aryl hydrocarbon receptor antagonist. In some embodiments, the culture medium and hydrogel do not comprise fibronectin and/or fragments thereof. In some embodiments, the culture medium and hydrogel do not comprise exogenous feeder cells. In some embodiments, the culture medium and hydrogel do not comprise a Notch ligand. In some embodiments, a Notch ligand is attached to the hydrogel.

In some embodiments, the HSPCs have been genetically modified. In some embodiments, the HSPCs have been genetically modified to introduce a wild type version of a gene into the genome of at least some of the HSPCs.

In some embodiments, the hydrogel expanded HSPCs comprise about 25% to about 95% HSPCs, about 50% to about 95% HSPCs or about 75% to about 95% HSPCs. In some embodiments, at least some of the HSCs in the HSPCs are quiescent.

In some embodiments, the poly-lysine is poly-L-lysine.

In some embodiments, the hydrogel expanded HSPC population comprises at least about 5%, at least about 10% or at least about 15% HSCs, or at least about 5%, at least about 10% or at least about 15% HSCs and MPPs.

In some embodiments, the hydrogel expanded HSPC population comprises at least 10 times the number of HSCs than the number of HSCs in the enriched HSPC population.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 depicts the expansion of TNC (Total nucleated cells; black bar), CD34⁺ cells (white bar) and HSCs and MPPs (Lin⁻ CD34⁺ CD38⁻ CD45RA⁻ CD133⁺ CD90^{+/-} cells; grey bar) in the four different culture systems, 3D ALG culture, 3D ALG+PLL culture in static flasks and roller bottles and 2D+Notch ligand culture for 14 days.

FIG. 2 depicts colony forming efficiency of cells in cell samples collected pre-culture (Day 0 (DO) and uncultured) and cultured in the following formats for 14 days: 2D+Notch culture, 3D ALG and 3D ALG+PLL static flasks and 3D ALG+PLL roller bottles. BFU-E: burst forming unit-erythroid colony; CFU-G/M/GM: colony forming unit (granulocyte, monocyte); CFU-GEMM: colony forming unit (granulocyte, erythrocyte, monocyte, megakaryocyte).

FIG. 3 depicts the expansion of TNC (Total nucleated cells; black bar), CD34⁺ cells (white bar) and HSCs/MPPs (Lin⁻ CD34⁺ CD38⁻ CD45RA⁻ CD133⁺ CD90^{+/-} cells; grey bar) expanded from adult mobilized peripheral blood derived CD34⁺ cells in two different culture systems; 3D ALG+PLL culture in static flasks and 2D+Notch ligand, and two different media formulations (StemSpan Serum Free Expansion Medium II (SFEMII); Stem Cell Technologies and StemPro HSC Expansion Medium Prototype; Thermo Fisher Scientific) for 14 days.

FIG. 4 depicts colony forming efficiency of CD34⁺ cells expanded from adult mobilized peripheral blood derived CD34⁺ cells in 2D Notch culture in SFEM II medium or StemPro medium and 3D ALG+PLL culture in SFEM II medium or StemPro medium for 14 days. BFU-E: burst forming unit-erythroid colony; CFU-G/M/GM: colony forming unit (granulocyte, monocyte); CFU-GEMM: colony forming unit (granulocyte, erythrocyte, monocyte, megakaryocyte).

### DETAILED DESCRIPTION

The present disclosure provides compositions and methods for culturing HSPC populations to maintain or increase the percentage of HSCs in an expanded HSPC population. An expanded HSPC population is generally prepared by isolating HSPCs (such as CD34⁺ and/or CD133⁺ cells), in which red blood cells, T cells, and other non-HSPCs have been depleted, to form enriched HSPCs (also referred to as an enriched HSPC population), and then expanding the HSPCs within a zwitterionic hydrogel comprising alginate and poly-lysine to form hydrogel expanded HSPCs (also referred to as an expanded HSPC population). In some embodiments, the hydrogel expanded HSPCs comprise a significant percentage of HSCs. In some embodiments, the hydrogel expanded HSPCs comprise a significant percentage of HSCs and MPPs. (As used herein, multipotent progenitors (MPPS) are Lin⁻ CD34⁺ CD38⁻ CD133⁺ CD45RA⁻ CD90- and HSCs are Lin⁻ CD34⁺ CD38⁻ CD133⁺ CD45RA⁻ CD90⁺.) In some embodiments, the hydrogel expanded HSPCs comprise a subpopulation of HSCs that, as a percentage of the total HSPCs, are the same or greater than the percentage of HSCs in the enriched HSPCs. In some embodiments, the hydrogel expanded HSPCs comprise a subpopulation of HSCs and MPPs that, as a percentage of the total HSPCs, are the same or greater than the percentage of HSCs and MPPs in the enriched HSPCs.

The hydrogel expanded HSPC populations can be used in cord blood transplantation, in immunotherapy, in gene therapy, and/or in preparation of other cells types derived from the HSPCs.

The HSPCs can be from a single source (e.g., a single human donor) or from multiple sources, e.g., from at least two or at least four different human donors. In some embodiments, the HSPCs are from umbilical cord blood and/or placental blood. In some embodiments, the HSPCs are somatic stem cells from a human after birth. In some embodiments, the HSPCs are from adult somatic stem cells. In some embodiments, the HSPCs are CD34⁺ HSPCs. In some embodiments, the HSPCs are CD133⁺ HSPCs.

In some embodiments, the enriched HSPCs comprise from about 50% up to about 95% HSPCs, from about 50% up to about 90% HSPCs, from about 60% up to about 90% HSPCs, from about 70% up to about 95% HSPCs, from about 70% up to about 90% HSPCs, from about 80% up to about 90% HSPCs. or from about 80% up to about 95% HSPCs.

In some embodiments, the hydrogel expanded HSPCs comprise from about 70% up to about 95% HSPCs, from about 75% up to about 95% HSPCs, from about 80% up to about 95% HSPCs, from about 70% up to about 90% HSPCs, from about 75% up to about 90% HSPCs, or from about 80% up to about 90% HSPCs.

In some embodiments, the percentage of HSCs in the HSPC population increases during the culturing step, such that the hydrogel expanded HSPC population comprises a higher percentage of HSCs, as compared to the enriched HSPCs. Typically, HSCs comprises less than 5% of the enriched HSPC population. In some embodiments of the present description, the percentage of HSCs increases by 1.5 fold (150%), 2 fold (200%), 2.5 fold (250%) or more, over the original percentage of HSCs in the enriched HSPCs (100%; wherein 100% HSCs in the enriched HSPC population comprise approximately equal to or less than 5% of the enriched HSPC population).

In some embodiments, the number of HSCs in the encapsulated enriched HSPC population increases during the culturing step, such that the hydrogel expanded HSPC population comprises a higher number of HSCs, as compared to the encapsulated enriched HSPCs. In some embodiments of the present description, the number of HSCs increases by at least 5 fold, at least 10 fold, at least 20 fold, at least 40 fold, at least 50 fold, or more, over the original number of HSCs in the encapsulated enriched HSPC population.

In some embodiments, the hydrogel expanded HSPC population comprises a greater proportion of quiescent cells as compared to the enriched HSPCs. In particular embodiments, a quiescent cell is a cell that is reversibly in the G0 phase of the cell cycle. That is, a quiescent cell is a cell that is in the G0 phase but is able to enter the cell cycle again. In contrast, a cell may enter the G0 phase of the cell cycle irreversibly, for example, through senescence or differentiation. The hydrogel expanded HSPC population of the present disclosure does not comprise a significant fraction of HSPCs that are senescent or differentiated.

In some embodiments, a hydrogel expanded HSPC population comprises a higher proportion of cells in the resting, non-cycling state (G0 phase) as compared to the enriched HSPCs. Resting, non-cycling cells can be identified with, for example, an antibody specific for a protein associated with cell proliferation such as, for example, anti-Ki-67 antibody, and a stain that can distinguish whether a cell is resting (non-cycling) or in cell division, such as, for example, Hoechst 33342. Ki-67 is a nuclear protein associated with cell proliferation. Resting, non-cycling cells (G0 phase) have little to no Ki-67 expression. Hoechst 33342 stain binds to nucleic acids, which are in higher abundance in cells in non-resting cells (e.g., G2-S/M), as compared to cells in G0 or G1.

In some embodiments, the hydrogel expanded HSPC population comprises a higher proportion of cells in the resting, non-cycling state (G0 phase) that are HSCs (Lin⁻ CD34⁺ CD38⁻ CD133⁺ CD45RA⁻ CD90⁺) as compared to the enriched HSPCs. In some embodiments, the hydrogel expanded, encapsulated HSPC population comprises a higher proportion of cells in the resting, non-cycling state (G0 phase) that are HSCs (Lin⁻ CD34⁺ CD38⁻ CD133⁺ CD45RA⁻ CD90⁺) as compared to the enriched, encapsulated HSPCs, at the beginning of culture.

In some embodiments, a hydrogel expanded HSPC population contains less than 2% CD3⁺ cells. In some embodiments, the hydrogel expanded HSPC population contains less than 1% CD3⁺ cells. In some embodiments, a hydrogel expanded HSPC population contains less than 2% CD19⁺ cells. In some embodiments, the hydrogel expanded HSPC population contains less than 1% CD19⁺ cells. In some embodiments, a hydrogel expanded HSPC population contains less than 20% CD34- cells. In some embodiments, the hydrogel expanded HSPC population contains less than 15% CD34- cells. In some embodiments, the hydrogel expanded HSPC population contains less than 10% CD34- cells. In some embodiments, a hydrogel expanded HSPC population contains less than 20% CD133- cells. In some embodiments, the hydrogel expanded HSPC population contains less than 15% CD133⁻ cells. In some embodiments, the hydrogel expanded HSPC population contains less than 10% CD133⁻ cells.

### Preparation and Culturing of HSPCs

The hydrogel expanded HSPCs comprise hematopoietic stem and progenitor cells that have been cultured from enriched HSPCs (also referred to as an enriched population of HSPCs or enriched HSPC population). The HSPCs are typically derived from one or more human sources. In some embodiments, the HSPCs are enriched for CD34⁺ HSPCs. In some embodiments, the enriched HSPCs are CD34⁺ hematopoietic stem and progenitor cells from a single human umbilical cord blood source or placental blood source. In some embodiments, the enriched HSPCs are CD34⁺ hematopoietic stem and progenitor cells from multiple human umbilical cord blood sources and/or placental blood sources. The HSPCs can comprise a single or multiple HLA-types because the HSPCs are not HLA-matched to each other, or a recipient prior to pooling. In some embodiments, the enriched HSPCs are CD34⁺ hematopoietic stem and progenitor cells from somatic stem cells from a single human PBMC source. In some embodiments, the enriched HSPCs are CD34⁺ hematopoietic stem and progenitor cells from somatic stem cells from multiple human PBMC sources. In some embodiments, the enriched HSPCs are depleted of T cells. As used herein, depleted of T cells refers to less than 2% CD3⁺ cells, or less than 1% CD3⁺ cells, or less than 0.5% CD3⁺ cells, or less than 0.1% CD3⁺ cells.

In some embodiments, the HSPCs are enriched for CD133⁺ HSPCs. In some embodiments, the enriched HSPCs are CD133⁺ hematopoietic stem and progenitor cells from a single human umbilical cord blood source or placental blood source. In some embodiments, the enriched HSPCs are CD133⁺ hematopoietic stem and progenitor cells from multiple human umbilical cord blood sources and/or placental blood sources. The HSPCs can comprise a single or multiple HLA-types because the HSPCs are not HLA-matched to each other, or a recipient prior to pooling. In some embodiments, the enriched HSPCs are CD133⁺ hematopoietic stem and progenitor cells from somatic stem cells from a single human PBMC source. In some embodiments, the enriched HSPCs are CD133⁺ hematopoietic stem and progenitor cells from somatic stem cells from multiple human PBMC sources. In some embodiments, the enriched HSPCs are depleted of T cells. As used herein, depleted of T cells refers to less than 2% CD3⁺ cells, or less than 1% CD3⁺ cells, or less than 0.5% CD3⁺ cells, or less than 0.1% CD3⁺ cells.

In some embodiments, the hydrogel expanded HSPCs derived from the enriched HSPCs are typically derived from one or more human sources. In some embodiments, the hydrogel expanded HSPCs are expanded CD34⁺ HSPCs. In some embodiments, the hydrogel expanded HSPCs are CD34⁺ hematopoietic stem and progenitor cells from a single human umbilical cord blood source or placental blood source. In some embodiments, the resulting hydrogel expanded HSPCs are CD34⁺ hematopoietic stem and progenitor cells from multiple human umbilical cord blood sources and/or placental blood sources. In some embodiments, the hydrogel expanded HSPCs can comprise a single or multiple HLA-types because the HSPCs are not HLA-matched to each other, or to a recipient prior to pooling. In some embodiments, the hydrogel expanded HSPCs are CD34⁺ hematopoietic stem and progenitor cells from a single somatic stem cell source (e.g., PBMCs). In some embodiments, the hydrogel expanded HSPCs are CD34⁺ hematopoietic stem and progenitor cells from multiple human somatic stem cell sources (e.g., PBMCs). In some embodiments, the hydrogel expanded HSPCs are depleted of T cells. As used herein, depleted of T cells refers to less than 2% CD3⁺ cells, or less than 1% CD3⁺ cells, or less than 0.5% CD3⁺ cells, or less than 0.1% CD3⁺ cells.

In some embodiments, the hydrogel expanded HSPCs are expanded CD133⁺ HSPCs. In some embodiments, the hydrogel expanded HSPCs are CD133⁺ hematopoietic stem and progenitor cells from a single human umbilical cord blood source and/or placental blood source. In some embodiments, the resulting hydrogel expanded HSPCs are CD133⁺ hematopoietic stem and progenitor cells from multiple human umbilical cord blood sources and/or placental blood sources. In some embodiments, the hydrogel expanded HSPCs can comprise a single or multiple HLA-types because the HSPCs are not HLA-matched to each other, or a recipient prior to pooling. In some embodiments, the hydrogel expanded HSPCs are CD133⁺ hematopoietic stem and progenitor cells from a single somatic stem cell source (e.g., PBMCs). In some embodiments, the hydrogel expanded HSPCs are CD133⁺ hematopoietic stem and progenitor cells from multiple human somatic stem cell sources (e.g., PBMCs). In some embodiments, the hydrogel expanded HSPCs are depleted of T cells. As used herein, depleted of T cells refers to less than 2% CD3⁺ cells, or less than 1% CD3⁺ cells, or less than 0.5% CD3⁺ cells, or less than 0.1% CD3⁺ cells.

HSPCs Derived From Umbilical Cord Blood and/or Placental Blood

The enriched HSPCs are typically CD34⁺ hematopoietic stem and progenitor cells or CD133⁺ hematopoietic stem and progenitor cells and are derived from one or more human sources. In some embodiments, the enriched HSPCs are CD34⁺ hematopoietic stem and progenitor cells from a single human umbilical cord blood source or placental blood source. In some embodiments, the enriched HSPCs are CD34⁺ hematopoietic stem and progenitor cells from multiple human umbilical cord blood sources and/or placental blood sources. The enriched HSPCs can comprise a single or multiple HLA-types because the HSPCs are not HLA-matched to each other, or a recipient prior to pooling. In some embodiments, the enriched HSPCs are CD133⁺ hematopoietic stem and progenitor cells from a single human umbilical cord blood source or placental blood source. In some embodiments, the enriched HSPCs are CD133⁺ hematopoietic stem and progenitor cells from multiple human umbilical cord blood sources and/or placental blood sources. The enriched HSPCs can comprise a single or multiple HLA-types because the HSPCs are not HLA-matched to each other, or a recipient prior to pooling. In some embodiments, the enriched HSPCs are depleted of T cells. As used herein, depleted of T cells refers to less than 2% CD3⁺ cells, or less than 1% CD3⁺ cells, or less than 0.5% CD3⁺ cells, or less than 0.1% CD3⁺ cells.

In some embodiments, the hematopoietic stem and progenitor cells are derived from cord blood and/or from placental blood (human cord blood and/or human placental blood). Such blood can be obtained by methods known in the art. See, e.g., U.S. Pat. Nos. 5,004,681 and 7,147,626 and U.S. Patent Application Publication No. 2013/0095079 (both incorporated herein by reference in their entirety) for a discussion of collecting cord and placental blood at the birth of a human. Umbilical cord blood and/or human placental blood collections are typically made under sterile conditions. Upon collection, cord or placental blood can be mixed with an anticoagulant, such as CPD (citrate-phosphate-dextrose), ACD (acid citrate-dextrose), Alsever's solution (Alsever et al., N.Y. St. J. Med. 41:126, 1941), De Gowin's Solution (De Gowin, et al., J. Am. Med. Ass. 114:850, 1940), Edglugate-Mg (Smith, et al., J. Thorac. Cardiovasc. Surg. 38:573, 1959), Rous-Turner Solution (Rous and Turner, J. Exp. Med. 23:219, 1916), other glucose mixtures, heparin, ethyl biscoumacetate, and the like. See, generally, Hum, 1968, Storage of Blood, Academic Press, New York, pp. 26-160. In one embodiment, ACD can be used.

Cord blood can preferably be obtained by direct drainage from the umbilical cord and/or by needle aspiration from the delivered placenta at the root and at distended veins. Preferably, the collected human cord blood and/or placental blood is free of contamination (e.g., bacterial or viral) and viral contamination in particular.

Prior to collection of the cord blood, maternal health history may be determined to identify risks that the cord blood cells might pose, e.g., transmitting genetic or infectious diseases, such as cancer, cancer (e.g., a leukemia), immune disorders, neurological disorders, hepatitis or AIDS. The collected cord blood can have undergone testing for one or more of cell viability, HLA typing, ABO/Rh typing, CD34⁺ cell count, CD133⁺ cell count, and/or total nucleated cell count.

After the umbilical cord blood and/or placental blood is collected from human donors at birth, the blood is processed to produce enriched HSPCs. In some embodiments, the HSPCs are preferably CD34⁺ cells or predominantly CD34⁺ HSPCs. In some embodiments, the HSPCs are preferably CD133⁺ cells or predominantly CD133⁺ HSPCs. The HSPCs are typically depleted of T cells and of red blood cells, resulting in enriched HSPCs. As used herein, depleted of T cells refers to less than about 2% CD3⁺ cells, less than about 1% CD3⁺ cells, or less than 0.5% CD3⁺ cells, or less than 0.1% CD3⁺ cells. Enrichment thus refers to a process wherein the percentage of HSPCs in the cell population is increased (relative to the percentage in the population before the enrichment procedure). Purification, such as CD34⁺ selection or CD133⁺ selection, is one example of enrichment and depletion of T cells and of red blood cells.

Prior to processing for enrichment, the collected cord and/or placental blood can be fresh or can have been previously cryopreserved. Any suitable technique known in the art for cell separation/selection can be used to carry out the enrichment for HSPCs. Methods which rely on differential expression of cell surface markers can be used. For example, cells expressing the cell surface marker CD34 can be positively selected using a monoclonal antibody to CD34, such that cells expressing CD34 are separated from cells not expressing CD34. Similarly, cells expressing the cell surface marker CD133 can be positively selected using a monoclonal antibody to CD133, such that cells expressing CD133 are separated from cells not expressing CD133. Moreover, the separation technique employed preferably maximizes the viability of the cells to be selected. The particular technique employed will depend upon efficiency of separation, cytotoxicity of the methodology, ease and speed of performance, and necessity for sophisticated equipment and/or technical skill.

Procedures for separation can include magnetic separation, using for example and not limitation, antibody-coated magnetic beads, affinity chromatography, and “panning” with antibody attached to a solid matrix, e.g., a plate, beads, and the like, or other convenient technique. Techniques providing accurate separation/selection include fluorescence activated cell sorters, which can have varying degrees of sophistication, e.g., a plurality of color channels, low angle and obtuse light scattering detecting channels, impedance channels, and the like.

The antibodies to cell surface molecules used in the selection process can be conjugated with a number of different materials adapted to the chosen selection process. For example, the antibody can be conjugated to magnetic beads, which allow for direct separation; biotin, which can be removed with avidin, streptavidin, or an antibody specific for biotin bound to, for example, a solid support; fluorochromes, which can be used with, for example, a fluorescence activated cell sorter; and the like, to allow for ease of separation of the particular cell type. Any cell separation technique can be employed which is not unduly detrimental to the viability of the remaining cells.

In a preferred embodiment, fresh cord blood units or frozen and thawed cord blood units are processed to enrich for CD34⁺ HSPCs using anti-CD34 antibodies directly or indirectly conjugated to magnetic particles in connection with a magnetic cell separator, for example, the CliniMACS® Cell Separation System (Miltenyi Biotec, Bergisch Gladbach, Germany), which employs nano-sized super-paramagnetic particles composed of iron oxide and dextran coupled to specific monoclonal antibodies. The CliniMACS® Cell Separator is a closed sterile system, outfitted with a single-use disposable tubing set. The disposable tubing set can be used for, and discarded after, processing a single unit of collected cord and/or placental blood to enrich for CD34⁺ HSPCs.

In a preferred embodiment, fresh cord blood units or frozen and thawed cord blood units are processed to enrich for CD133⁺ HSPCs using anti-CD133 antibodies directly or indirectly conjugated to magnetic particles in connection with a magnetic cell separator, for example, the CliniMACS® Cell Separation System (Miltenyi Biotec, Bergisch Gladbach, Germany), which employs nano-sized super-paramagnetic particles composed of iron oxide and dextran coupled to specific monoclonal antibodies. The CliniMACS® Cell Separator is a closed sterile system, outfitted with a single-use disposable tubing set. The disposable tubing set can be used for, and discarded after, processing a single unit of collected cord and/or placental blood to enrich for CD133⁺ HSPCs.

In an embodiment, a single umbilical cord blood and/or placental blood unit is used to prepare enriched HSPCs. In other embodiments, two or more or four or more umbilical cord blood and/or placental blood units can be pooled prior to enriching for HSPCs. In another embodiment, individual populations of HSPCs can be pooled after enriching for the HSPCs. In specific embodiments, the number of umbilical cord blood and/or placental blood units, or populations of HSPCs, that are pooled is 2, 4, 5, 6, 7, 8, 9, 10, 15, 20, 25, 30, 35, or 40, or at least any of the foregoing numbers. In some embodiments, the pool contains 2 to 8, 4 to 8, 2 to 10, 4 to 10, 4 to 20, or 4 to 25, and no more than 20 or 25, umbilical cord blood and/or placental blood units, or HSPC populations. Pooling is typically done to increase the number of HSPCs in a cell product which can be frozen and stored prior to administration to a recipient.

The umbilical cord blood or hematopoietic stem or stem and progenitor cell populations can be pooled with or without regard to the HLA-type of the HSPCs. In some embodiments, the cells in the pool are combined without regard to race or ethnicity. In some embodiments, the cells in the pool are derived from the umbilical cord blood and/or placental blood of individuals of the same race, e.g., African-American, Caucasian, Asian, Hispanic, Native-American, Australian Aboriginal, Inuit, Pacific Islander, or derived from umbilical cord blood and/or placental blood of individuals of the same ethnicity, e.g., Irish, Italian, Indian, Japanese, Chinese, Russian, and the like.

Typically, prior to enrichment for HSPCs, the red blood cells and white blood cells of the cord blood or placental blood are separated. In some embodiments, depletion of red blood cells refers to separation of red blood cells from white blood cells. Once the separation of the red blood cells and the white blood cells has taken place, the red blood cell fraction can be discarded, and the white blood cell fraction can be processed, for example, in the magnetic cell separator as described above to enrich for CD34⁺ HSPCs or CD133⁺ HSPCs. Separation of the white and red blood cell fractions can be performed by any method known in the art, including for example and not limitation, apheresis, centrifugation techniques, and the like. Other separation methods that can be used include the use of commercially available products FICOLL™ or FICOLL-PAQUE™ or PERCOLL™ (GE Healthcare, Piscataway, N.J.). FICOLL-PAQUE™ is normally placed at the bottom of a conical tube, and the whole blood is layered above. After being centrifuged, the following layers will be visible in the conical tube, from top to bottom: plasma and other constituents, a layer of mono-nuclear cells called buffy coat containing the mononuclear cells (white blood cells), FICOLL-PAQUE™, and erythrocytes and granulocytes, which should be present in pellet form. This separation technique allows easy harvest of the mononuclear cells. Another method includes Hetastarch (hydroxyl-ethyl starch)-mediated sedimentation for removal of red blood cells, which can be used for cord blood and can also be used for bone marrow (BM) sources.

Optionally, prior to CD34⁺ or CD133⁺ cell selection, an aliquot of the cord blood or placental unit can be checked for total nucleated cell count and/or CD34⁺ or CD133⁺ cell content. In a specific embodiment, after the CD34⁺ cell selection, both CD34⁺ and CD34- cell fractions are recovered. Optionally, DNA can be extracted from a sample of the unprocessed blood product or CD34- cell fraction for initial HLA typing and future chimerism studies. Similarly, in a specific embodiment, after the CD133⁺ cell selection, both CD133⁺ and CD133⁻ cell fractions are recovered. Optionally, DNA can be extracted from a sample of the unprocessed blood product or CD133⁻ cell fraction for initial HLA typing and future chimerism studies.

HSPCs from Somatic Stem Cells

In some embodiments, HSPCs are derived from a human or humans after birth. In some embodiments, enriched HSPCs are CD34⁺ hematopoietic stem and progenitor cells or CD133⁺ hematopoietic stem and progenitor cells and are derived from one or more human sources. In some embodiments, the enriched HSPCs are CD34⁺ hematopoietic stem and progenitor cells from a single human somatic stem cell source. In some embodiments, the enriched HSPCs are CD34⁺ hematopoietic stem and progenitor cells from multiple human somatic stem cell sources. The enriched HSPCs can comprise a single or multiple HLA-types because the HSPCs are not HLA-matched to each other prior to pooling. In some embodiments, the enriched HSPCs are CD133⁺ hematopoietic stem and progenitor cells from a single human somatic stem cell source. In some embodiments, the enriched HSPCs are CD133⁺ hematopoietic stem and progenitor cells from multiple human somatic stem cell sources. The enriched HSPCs can comprise a single or multiple HLA-types because the HSPCs are not HLA-matched to each other, or a recipient prior to pooling. In some embodiments, the enriched HSPCs are depleted of T cells. As used herein, depleted of T cells refers to less than 2% CD3⁺ cells, or less than 1% CD3⁺ cells, or less than 0.5% CD3⁺ cells, or less than 0.1% CD3⁺ cells.

In some embodiments, HSPCs are from somatic stem cells collected from PBMCs after birth. (e.g., an adult or a child). PBMCs can be obtained from a subject using any suitable methodology, such as via mobilization of the subject's stem cells into the peripheral blood using a mobilizer, aspiration of the bone marrow, and/or apheresis, such as leukapheresis.

In some embodiments, PBMCs are obtaining from a subject following mobilization of stem cells into the peripheral blood using a mobilizer, followed by leukapheresis. As used herein, a “mobilizer” refers to any substance, whether it is a small organic molecule, synthetic or naturally derived, or a polypeptide, such as a growth factor or colony-stimulating factor or an active fragment or mimic thereof, a nucleic acid, a carbohydrate, an antibody, or any other agent that acts to enhance the migration of stem cells from the BM into the peripheral blood. Such a “mobilizer” can increase the number of stem cells (e.g., hematopoietic stem cells or hematopoietic stem and progenitor cells) in the peripheral blood, thus allowing for a more accessible source of stem cells. Any mobilizer suitable for increasing the number of stem cells in the subject that are available to be harvested can be utilized. In an embodiment, the mobilizer is a cytokine such as granulocyte colony-stimulating factor (G-CSF). A commercial example of a mobilizer suitable is NEUPOGEN™ (filgrastim). Another example of a mobilizer suitable for use in the present disclosure is a recombinant methionyl human stem cell factor which is commercially available as STEMGEN™. Yet another example of a mobilizer suitable for use in the present disclosure is plerixafor which is an inhibitor of the CXCR4 chemokine receptor and blocks binding of its cognate ligand, stromal cell-derived factor-1a (SCF-1 alpha) and is commercially available as MOZOBIL™ from Genzyme. In some embodiments, PBMCs are isolated from a subject following BM harvest with a target collection goal of 15 cc/kg body weight or were administered daily G-CSF (filgrastim; 16 mg/kg BID; days 1-6) and plerixafor (240 mg/kg/day; days 4-6) subcutaneously to mobilize CD34⁺ cells, followed by leukapheresis.

In some embodiments, HSPCs are isolated from the PBMCs by enrichment of CD34⁺ cells, such as by methods described herein or as known to a person of skill in the art, to prepare enriched HSPCs. In some embodiments, CD34 cells are enriched by lineage depletion as described in Adair et al., (Haematologica 103(11):1806-1814, 2018; the disclosure of which is incorporated herein by reference).

### Enriched HSPCs

The enriched HSPCs as described above can be subsequently processed prior to expansion, for example, by suspension in an appropriate cell culture medium or cryopreservation for storage and/or transport. In a preferred embodiment, the cell culture medium or cryopreservation medium is suitable for the maintenance of viability of HSPCs. For example, the cell culture medium can be a serum free, serum component free (for example, human serum albumin, or human platelet lysate), cytokine free hematopoietic stem cell or stem and progenitor cell culture medium, such as for example, StemSpan™ SFEM, StemSpan™ SFEM II, StemSpan™-ACF, Stemline I, Stemline II, StemMACS™, X-VIVO 10, STEMium®, StemPro-34 SFM, StemPro HSC media (Prototype; Life Technologies), PRIME-XV®, minimal essential media (MEM), Eagles' minimal essential medium (EMEM), Dulbecco's Modified Eagle Media (DMEM), Ham's Nutrient Mixtures (Ham's F-10, and Ham's F-12), Roswell Park Institute Medium (RPMI), Iscove's Modified Dulbecco's Medium and (IMDM), or their combinations. Growth factors are added to the cell culture medium. For example, in some embodiments, growth factors are added at the following concentrations: about 50-300 ng/ml of stem cell factor (SCF), about 50-300 ng/ml of Flt3-ligand (Flt3-L), about 50-100 ng/ml of thrombopoietin (TPO), about 50-100 ng/ml of interleukin-6 (IL-6), and about 10-50 ng/ml of interleukin-3 (IL-3). In more specific embodiments, the cell culture medium contains 300 ng/ml of stem cell factor, 300 ng/ml of Flt3-Ligand, 100 ng/ml of thrombopoietin, 100 ng/ml of interleukin-6 and 10 ng/ml of interleukin-3; or 50 ng/ml of stem cell factor, 50 ng/ml of Flt3-Ligand, 50 ng/ml of thrombopoietin, 50 ng/ml of interleukin-6 and 10 ng/ml of interleukin-3. In another preferred embodiment, the cell culture medium includes, or alternatively consists of, a serum free, hematopoietic stem cell or stem and progenitor cell culture medium (e.g., STEMSPAN™ Serum Free Expansion Medium or STEMSPAN™ Serum Free Expansion Medium II (StemCell Technologies, Vancouver, British Columbia)) supplemented with 10 ng/ml recombinant human Interleukin-3 (rhIL-3), 50 ng/ml recombinant human Interleukin-6 (rhIL-6), 50 ng/ml recombinant human Thrombopoietin (rhTPO), 50 ng/ml recombinant human Flt3-Ligand (rhFlt3-L), 50 ng/ml and recombinant human stem cell factor (rhSCF). In another preferred embodiment, the cell culture medium consists of a serum-free hematopoietic stem cell or stem and progenitor cell culture medium (e.g., StemSpan Serum Free Expansion Medium II (SFEM II, StemCell Technologies, Vancouver, British Columbia)) supplemented with recombinant human rhSCF, rhFlt3-L, rhTPO, rhIL-6 (each at 50 ng/ml final concentration), and rhIL-3 (at 10 ng/ml final concentration). The cryopreservation medium can be any suitable medium including any of those described below.

In a specific embodiment, the HSPCs are red blood cell depleted, and the number of CD34⁺ and/or CD133⁺ cells in the red blood cell depleted fraction is determined. In some embodiments, depletion of red blood cells refers to separation of red blood cells from white blood cells or separation of red blood cells from CD34⁺ and/or CD133⁺ cells. Preferably, umbilical cord blood and/or placental blood units containing more than 3.5 million CD34⁺ cells are subject to the enrichment methods described above.

After the HSPCs have been isolated (e.g., from human cord blood and/or human placental blood collected from humans at birth or from humans after birth) according to the enrichment methods described above or other methods known in the art, the enriched HSPCs are encapsulated in a hydrogel.

The enriched HSPCs are typically a suspension of single cells of HSPCs that are then encapsulated within an alginate-poly-lysine hydrogel. The poly-lysine is typically poly-L-lysine. The encapsulated HSPCs are capable of expanding without substantially differentiating within the microenvironment of the hydrogel. In some embodiments, the encapsulated HSPCs can expand without substantially differentiating within the microenvironment of the hydrogel, such that the percentage of HSCs is maintained or increased in the hydrogel expanded HSPCs, relative to the starting enriched HSPCs. The alginate encapsulation provides a scalable and reversible culture environment useful to maintain and expand the HSPCs without significant differentiation of HSCs into progenitors (e.g., lineage committed progenitors and/or terminally differentiated cell types such as, for example, CD3⁺ T cells, CD56⁺ NK cells, macrophage, CD20⁺ B cells, CD14⁺ monocytes, CD15⁺ neutrophils).

In some embodiments, the alginate poly-lysine hydrogel further includes a divalent cation. Suitable divalent cations include Ca₂⁺ or Ba₂⁺.

In some embodiments, the alginate concentration is from about 1% (w/v) to about 3% (w/v) during encapsulation. In some embodiments, the alginate concentration is from about 1% (w/v) to about 5% (w/v) during encapsulation. In some embodiments, the alginate concentration is about 2% (w/v) during encapsulation.

In some embodiments, the poly-lysine concentration is from about 0.01% (w/v) to about 10% (w/v) during encapsulation. In some embodiments, the poly-lysine concentration is about 0.1% (w/v) during encapsulation. In some embodiments, the poly-lysine is poly-L-lysine and the concentration is from about 0.01% (w/v) to about 10% (w/v) during encapsulation. In some embodiments, the poly-lysine is poly-L-lysine and the concentration is about 0.1% (w/v) during encapsulation.

In some embodiments, the HSPCs are encapsulated at a cell seeding density of about 1×10⁴ cells/ml to about 1×10⁷ cells/ml. In some embodiments, the HSPCs are encapsulated at a cell seeding density of about 1×10⁵ cells/ml to about 1×10⁷ cells/ml. In some embodiments, the HSPCs are encapsulated at a cell seeding density of about 1×10⁶ cells/ml to about 1×10⁷ cells/ml. In some embodiments, the HSPCs are encapsulated at a cell seeding density of about 5×10⁶ cells/ml. In some embodiments, the HSPCs are encapsulated at a cell seeding density of about 1×10⁶ cells/ml to about 1×10⁷ cells/ml. In some embodiments, the HSPCs are encapsulated at a cell seeding density of about 5×10⁶ cells/ml.

Encapsulation of enriched HSPCs can be performed using any suitable technique. For example, techniques such as a coaxial electrospray method, centrifugal coating, a flow vibration nozzle, an oil-aqueous emulsion, an air dripping nozzle, pan coating, ionotropic gelling, coacervation-phase separation, interface crosslink or polymerization, spray-drying, and their combinations can also be used.

The alginate is typically an alginate produced in and/or under Good Manufacturing Practices (GMP) conditions, and the molecular weight can be selected from 1 kDa to 2000 kDa. The C-5 epimer-L-guluronate (G)/b-D-mannuronate (M) ratio can be selected from 0.1 to 10. The working viscosity of alginate solution can be selected from 1 mPa*s to 500 mPa*s. The alginate preferably has a low endotoxin, e.g., (EU/g)<100.

The alginate is primarily physically crosslinked by a gelling agent, such as, for example a positively charged polymer or a divalent ion such as Mg₂⁺, Ca₂⁺, Sr₂⁺, Ba₂⁺, or their combinations.

The positively charged polymers can be selected from, but not limited to poly-lysine, poly-L-lysine, poly-D-lysine, poly-histidine, poly-ornithine, cationic chitosan, cationic gelatin, cationic dextran, cationic cellulose, cationic cyclodextrin, polybrene, polyethyleneimine, polyvinyl pyridine, poly(diallyldimethylammonium chloride), poly(amidoamine)s, and poly(amino-co-ester), poly(2-N,N-dimethylaminoethylmethacrylate). The positively charged polymer is typically poly-lysine, poly-L-lysine, poly-D-lysine, or a mixture thereof.

Suitable solutions for the alginate and gelling agent(s) include, but are not limited to saline (e.g., PBS, DPBS, HEPES, HBSS, EBSS, citrate saline, and the like), cell culture medium (e.g., StemSpan™ SFEM, StemSpan™ SFEM II, StemSpan™-ACF, STEMLINE™ I, STEMLINE™ II, StemMACS™, X-VIVO™ 10, STEMium®, STEMPRO™-34 SFM, STEMPRO™ HSC media (Prototype; Life Technologies), PRIME-XV®, minimal essential media (MEM), Eagles' minimal essential medium (EMEM), Dulbecco's Modified Eagle Media (DMEM), Ham's Nutrient Mixtures (Ham's F-10, and Ham's F-12), Roswell Park Institute Medium (RPMI), Iscove's Modified Dulbecco's Medium and (IMDM)), or their combinations.

The formation of hydrogel encapsulated cells can be performed by several methods. These include the formation of both alginate/cell water droplets and CaCl₂ containing bath. Microcapsule technology has been described, for example in U.S. Pat. No. 8,435,787. Another method includes the formation of both alginate/cell water droplets and CaCl₂ containing water droplets within an oil phase. When the two types of droplets fuse together, a cell-containing cross-linked hydrogel bead is formed (see, e.g., H. Shintaku, et al., Microsystems Technology 13:951, 2007). This method is described more fully below.

There are two stages in the typical process; droplet formation, and the coalescence of droplets to form the hydrogel. For droplet formation, a droplet of sodium alginate solution containing cells is formed from a nozzle located upstream of a microchannel by introducing an aqueous phase into oil in the microchannel. The alginate droplet flows downstream in the main channel, following the flow of the continuous liquid phase. Secondly, the alginate droplet is fused with droplets of calcium chloride solution formed from a second nozzle located downstream.

In order to produce a hydrogel with beads smaller than 300 μm in diameter (e.g., beads within the range of about 10 μm to about 300 μm), the channel depth is preferably about 50 μm, with a preferred diameter of 50 μm for the nozzle and 200 μm for the main channel, respectively.

Sodium alginate solution is preferably employed at a concentration from about 1% to about 3% (w/v) or about 1.5% w/v, and cells dispersed in the alginate at a concentration of 10⁵ cells/ml. Calcium chloride is preferably provided at a concentration of 0.1 M. Vegetable oil such as sunflower oil can be used as the oil phase.

A second protocol has been described by Workman, et al., Macromolecular Rapid Communications 29:165, 2008. In this method, a shielded junction is employed to generate alginate microspheres. Aqueous sodium alginate mixed with CaCO₃ and cells are introduced into a central channel. Sunflower oil mixed with acetic acid is supplied to the outermost channels. Sunflower oil is supplied to the intermediate channels to act as a shield preventing the alginate solution from coming into contact with the acidified oil flow. Between the channels the two oils flow in a laminar fashion, with minimal diffusion of H⁺ into the protective sunflower oil. After droplet formation at the junction, H⁺ diffuses into the alginate droplet, thus liberating Ca₂⁺ from CaCO₃, which causes gelation of the alginate. Channels prior to the junction preferably have a cross-sectional area of about 500 μm², after the junction channels preferably about 1000 μm².

Encapsulation can also be performed using a jetting encapsulation technique. Many such techniques are known in the art; preferred are bio-electrospray jetting, aerodynamically assisted bio-jetting, and pressure-assisted cell jetting. Electrospraying is also known as bio-electrospraying or electrohydrodynamic jetting and relies on a potential difference between a spray nozzle or needle and a grounded electrode to produce droplets of defined size.

The media are passed through a conducting needle that is held at a higher potential than the electrode, setting up an external electric field into which the media exiting the needle are passed. Needles are hollow, having an internal diameter of between 0.2 and 2 mm, and either flat or chamfered edge geometries. Needles can also be coaxial, such that different fluids can be sprayed from the same needle contemporaneously. The formation of the droplets is determined by the potential difference (difference in voltage) between the needle and the electrode, the flow rate of the medium and its relative features such as viscosity, surface tension, electrical conductivity and relative permittivity. Voltage and distance are related as the electric field depends on both variables. Normally, encapsulations are done at 1 or 2 cm distance with voltages around 5-10 kV. When the jet is stable, near monodistributions of droplet sizes can be achieved. Living cells can be encapsulated using this technology (see, e.g., Jayasinghe et al., Small 2:216-219, 2006; and Jayasinghe et al., Biotechnol. J. 1:86-94, 2006). Although early studies resulted in unstable jets with a wide dispersion of droplet sizes, this was improved using a coaxial jetting needle to create stable jetting (Jayasinghe et al., Lab Chip 6:1086-1090, 2006) with the microencapsulation material sprayed in the outer jet and the biomaterial in the inner jet.

Aerodynamically assisted jetting relies on a pressure gradient. A pressure is created in a chamber, with respect to the surrounding atmosphere, which provides the drawing effect to create the jet. Living cells can be encapsulated in this way (Arumuganathar et al., Biomed. Mat. 2:158-168, 2007).

Pressure-assisted jetting employs a coaxial needle, where one orifice is used to jet the medium, and the second serves as the conduit for a pressure to be applied. Unlike aerodynamically assisted jetting, there is no pressurized chamber.

For a general review of jetting technologies, see Jayasinghe, Regen. Med. 3:49-61, 2008, as well as U.S. Pat. No. 6,649,384, U.S. Patent Application Publication No. 2006/0051329 and U.S. Pat. No. 4,353,888. (All incorporated herein by reference in their entirety). Jetting technologies can be scaled up by using multiple nozzles.

In some embodiments, in order to prepare the HSPCs for encapsulation, the HSPCs, which are typically individual cells, are washed in a suitable aqueous buffer such as PBS, precipitated and resuspended in a buffer comprising the encapsulating polymer. Encapsulation materials can be any suitable form of alginate, preferably a GMP alginate, and poly-lysine, preferably poly-L-lysine. The alginate is capable of ready solidification to form a matrix having the desired properties, and that is insoluble in water or saline at physiological pH. The desired properties include nutrient permeability. Typically, the resulting hydrogel is charge balanced. As used herein, a “charge balanced” hydrogel refers to a hydrogel that is formed of alginate and poly-lysine, in which the alginate and poly-lysine reach an equilibrium as the hydrogel capsules are formed.

In some embodiments, the alginate is preferably in a PBS buffer lacking calcium ions and magnesium ions; this prevents premature solidification of the encapsulating hydrogel. Typically, the buffer comprises 1-5% by weight of alginate in a suitable buffer, such as MOPS, PBS or a cell culture medium described herein.

The gelling agent for the hydrogel can be introduced in one of three manners: (1) a secondary droplet population is generated and induced to fuse with the cell capsules; (2) the cell droplets are extracted into a water phase stream containing the polymerization agent established parallel to the oil phase; (3) the gelling agents are dissolved directly into the oil phase; or (4) the cells and alginate are introduced into a solution of the gelling agent.

The gelling solution, e.g., preferably, Ca₂⁺, Sr₂⁺ or Ba₂⁺ ions, are used to solidify the alginate. CaCl₂, or the respective Sr or Ba compounds, are dissolved in water at a concentration of between 10 mM and 1 M. Combinations of Ca, Sr and Ba can be used with as little as 1 mM of one salt to achieve optimum hydrogel properties. The gelling solution typically includes a poly-lysine, such as poly-L-lysine, e.g., at a concentration of 0.015 to 0.10% (w/v). This solution is preferably held in a collection vessel, which is placed at the electrode of an electrospray unit. The HSPCs, suspended in the alginate solution, are passed through the spraying machine such that droplets are collected in the vessel which holds the solidifying or gelling solution. Encapsulated cells can be retrieved from the bottom of the vessel after spraying.

### Culture of Encapsulated HSPCs

The encapsulated HSPCs are cultured in a culture medium to maintain and/or expand the HSPCs, e.g., CD34⁺ HSPCs or CD133⁺ HSPCs (in some embodiments, collectively referred to as expansion). The HSPCs are cultured in a culture medium under conditions that maintain HSPC viability and/or HSPC proliferation (e.g., promoting mitosis such that the HSPCs grow and divide (proliferate)). In some embodiments, during culturing of the encapsulated HSPCs, minimal differentiation of HSCs into terminally differentiated cell types occurs (i.e., less than 5%, less than 4%, less than 3% or less than 2% of the resulting cells). In some embodiments, during culturing of the encapsulated HSPCs, the percentage of committed progenitors (e.g., myeloid and lymphoid) remains about the same as in the enriched HSPCs (e.g., less than 5% change, less than 10% change). In some embodiments, during culturing of the encapsulated HSPCs, minimal differentiation of HSCs and MPPs into terminally differentiated cell types occurs (i.e., less than 5%, less than 4%, less than 3% or less than 2% of the resulting cells). In some embodiments, during culturing of the encapsulated HSPCs, the percentage of committed progenitors (e.g., myeloid and lymphoid) remains about the same as the enriched HSPCs (e.g., less than 5% change, less than 10% change).

General culturing and expansion techniques include, but are not limited to, those described in U.S. Pat. No. 7,399,633; U.S. Patent Application Publication No. 2013/0095079; Delaney et al., Nature Med. 16(2):232-236, 2010 (all incorporated by reference in their entirety); as well as those described below. These techniques can be adapted for use according to the methods and compositions described herein.

As used herein, a “feeder cell layer”, “feeder layer” or “feeder cells” refer to exogenous cells of one type that are co-cultured with cells of a second type (e.g., HSPCs), to provide an environment in which the cells of the second type can be maintained and differentiate or proliferate. Without being bound by any theory, feeder cells can provide, for example, peptides, polypeptides, electrical signals, organic molecules, nucleic acid molecules, growth factors, other factors (e.g., cytokines), and metabolic nutrients to the second type of cells.

In some embodiments, the HSPCs are cultured in a culture medium which is serum free and suitable for maintaining viability of hematopoietic stem and progenitor cells, in the presence of growth factors, and are exposed to cell growth conditions (e.g., promoting mitosis) such that the HSPCs proliferate to generate an expanded population of HSPCs (an expanded HSPC population, expanded HSPCs, or alternatively, a hydrogel expended HSPC population or hydrogel expanded HSPCs), and maintain the viability of HSCs in the HSPC population.

In an exemplary embodiment, the culture medium, suitable for maintenance and/or expansion of hematopoietic stem and progenitor cells, is a serum free, culture medium such as Iscove's MDM containing non-animal sourced BSA, recombinant human insulin, human transferrin, 2-mercaptoethanol, and other supplements, with growth factors, as described herein. In other embodiments, the hematopoietic stem cell culture medium is STEMSPAN™ Serum Free Expansion Medium (StemCell Technologies, Vancouver, British Columbia), or STEMSPAN™ Serum Free Expansion Medium II (StemCell Technologies, Vancouver, British Columbia).

In some embodiments, the HSPCs are cultured in the absence of a Notch ligand (i.e., an agonist of Notch function effective to inhibit differentiation, also referred to as a Notch agonist). In some embodiments, the HSPCs are cultured in the absence of a Notch ligand and fibronectin and/or fragments thereof. In some embodiments, the HSPCs are cultured in the absence of a Notch ligand, fibronectin and/or fragments thereof, and an aryl hydrocarbon antagonist (e.g., SR1) and pyrimido-indole derivatives, such as UM729 and UM171. In some embodiments, the HSPCs are cultured in the presence of a Notch ligand, typically attached to a functionalized form of alginate and/or poly-lysine. In either embodiment, differentiation of HSPCs to further committed cell types is minimized during culturing. In some embodiments, differentiation of HSCs to multipotent progenitors, myeloid and lymphoid progenitors and more committed cell types is minimized during culturing. In some embodiments, the percentage of HSCs remains the same or increases during culturing. In some embodiments, differentiation of HSCs to myeloid and lymphoid progenitors and more committed cell types is minimized during culturing. In some embodiments, differentiation of HSCs and MPPs to myeloid and lymphoid progenitors and more committed cell types is minimized during culturing. In some embodiments, the percentage of HSCs and MPPs remains the same or increases during culturing.

In some embodiments, the HSPCs are cultured for 2, 3, 4, 5, 6, 7, 8, 9, 10, 11, 12, 13, 14, 15, 16, 17, 18, 19, 20, 21, 22, 23, 24, or 25 days or more; or, preferably, the HSPCs are cultured for at least 10 days, from about 2 to about 10 days, from about 2 to about 21 days, from about 7 to about 15 days, from about 7 to about 14 days, or from about 13 to about 15 days. In some embodiments, the HSPCs are cultured for about 14 days, about 13 days or about 15 days. Typically, the encapsulated HSPCs are cultured for a time period sufficient to maximize the cell density within the hydrogel capsule, but not for so long a time period that the hydrogel capsule bursts. Under the most common ex vivo cell culture conditions for HSPCs this is typically about 14 days; however, if a less typical medium or cell culture conditions are used this time period can be shortened or extended.

An exemplary culture condition for culturing the encapsulated HSPCs includes culturing the cells for 7 to 14 days or 7 to 15 days in a serum free, culture medium supplemented with the following human growth factors: stem cell factor, Flt3-Ligand, thrombopoietin, interleukin-6 and interleukin-3. Preferably, the foregoing growth factors are present at the following concentrations: 50-300 ng/ml stem cell factor, 50-300 ng/ml Flt3-Ligand, 50-100 ng/ml thrombopoietin, 50-100 ng/ml interleukin-6 and 10-50 ng/ml interleukin-3. In more specific embodiments, 300 ng/ml stem cell factor, 300 ng/ml of Flt3-Ligand, 100 ng/ml thrombopoietin, 100 ng/ml interleukin-6 and 10 ng/ml interleukin-3, or 50 ng/ml stem cell factor, 50 ng/ml of Flt3-Ligand, 50 ng/ml thrombopoietin, 50 ng/ml interleukin-6 and 10 ng/ml interleukin-3, are used. In another preferred embodiment, the culture medium (e.g., STEMSPAN™ Serum Free Expansion Medium (StemCell Technologies, Vancouver, British Columbia)) contains, or consists of, 10 ng/ml recombinant human Interleukin-3 (rhIL-3), 50 ng/ml recombinant human Interleukin-6 (rhIL-6), 50 ng/ml recombinant human Thrombopoietin (rhTPO), 50 ng/ml recombinant human Flt3-Ligand (rhFlt3-L), 50 ng/ml and recombinant human stem cell factor (rhSCF). In another preferred embodiment, the culture medium (e.g., StemSpan Serum Free Expansion Medium II (SFEM II, StemCell Technologies, Vancouver, British Columbia)) contains, or consists of, recombinant human rhSCF, rhFlt3-L, rhTPO, rhIL-6 (each at 50 ng/ml final concentration), and rhIL-3 (at 10 ng/ml final concentration).

In some embodiments, a Notch ligand is included in the culturing system. In some embodiments, the Notch ligand is attached to the hydrogel (e.g., alginate-poly-lysine hydrogel). In some embodiments, the Notch ligand is DXI (Delta1^{ext-IgG}) and the culturing is performed as described herein; the Delta1^{ext-IgG} (DXI) is attached to the hydrogel. In some embodiments, a Notch ligand is used and attached to an alginate-poly-lysine hydrogel, where the Notch ligand is a Notch 1 and/or Notch 2 receptor specific antibody (see, for example, Published U.S. Patent Application Publication No. 2017/0107493; incorporated herein by reference) and the culturing is performed as described herein: the Notch 1 and/or Notch 2 receptor specific antibody is attached to the hydrogel. Preferably, the culture medium (e.g., STEMSPAN™ Serum Free Expansion Medium or STEMSPAN Serum Free Expansion MediumII (StemCell Technologies, Vancouver, British Columbia)) is supplemented with 10 ng/ml recombinant human Interleukin-3 (rhIL-3), 50 ng/ml recombinant human Interleukin-6 (rhIL-6), 50 ng/ml recombinant human Thrombopoietin (rhTPO), 50 ng/ml recombinant human Flt3-Ligand (rhFlt3-L), 50 ng/ml and recombinant human stem cell factor (rhSCF).

In some embodiments, the culture medium does not include growth factors other than rhIL-3, rhIL-6, rhTPO, rhFlt3-L and rhSCF.

In some embodiments, the culture medium does not contain the following added growth factors or cytokines: IL-7, GM-CSF, G-CSF, LIF, MIP-1a, IL-2 or IL-15. In some embodiments, the culture medium does not contain an aryl hydrocarbon receptor antagonist, such as those described in U.S. Pat. No. 9,175,266 or U.S. Patent Application Publication No. 2018/0237749 (incorporated herein by reference in their entirety).

After culturing of the hematopoietic stem and progenitor cells, the total number of cells and viable HSPC's (e.g., CD34⁺ HSPCs or CD133⁺ HSPCs) can be determined. For example, at day 14 during culturing, a sample can be taken for determination of the total viable nucleated cell count. In addition, the total number of CD34⁺ cells and/or CD133⁺ cells can be determined by multi-parameter flow cytometry, and, thus, the percentage of CD34⁺ cells and/or CD133⁺ cells in the sample.

Viability can be determined by any method known in the art, for example, by trypan blue exclusion or 7-AAD exclusion. The percentage of viable HSPCs can be assessed by flow cytometry and use of a stain that is excluded by viable cells. The percentage of viable HSPCs is equal to the number of HSPC⁺ cells that exclude 7-AAD (or other appropriate stain) in an aliquot of the sample divided by the total cell number of HSPCs (TNC; both viable and non-viable) of the aliquot.

After culturing of the hematopoietic stem and progenitor cells, the percentage of HSCs in the expanded HSPCs can be determined. For example, at day 14 during culturing, a sample can be taken for determination of the HSCs as a percentage of total HSPCs by, for example, multi-parameter flow cytometry, where the CD34⁺ cells are HSPCs and HSCs are CD34⁺ CD38⁻ CD45RA⁻ CD133⁺ CD90⁺. Similarly, at day 14 during culturing, a sample can be taken for determination of the HSCs as a percentage of total HSPCs by, for example, multi-parameter flow cytometry, where the CD133⁺ cells are HSPCs and HSCs are CD34⁺ CD38⁻ CD45RA⁻ CD133⁺ CD90⁺. In some embodiments, the percentage of HSCs and MPPs in the expanded HSPCs can be determined. For example, at day 14 during culturing, a sample can be taken for determination of the HSCs and MPPs as a percentage of total HSPCs by, for example, multi-parameter flow cytometry, where the CD34⁺ cells are HSPCs, the HSCs are CD34⁺ CD38⁻ CD45RA⁻ CD133⁺ CD90⁺, and the MPPs are CD34⁺ CD38⁻ CD45RA⁻ CD133⁺ CD90⁻. Similarly, at day 14 during culturing, a sample can be taken for determination of the HSCs and MPPs as a percentage of total HSPCs by, for example, multi-parameter flow cytometry, where the CD133⁺ cells are HSPCs, the HSCs are CD34⁺ CD38⁻ CD45RA⁻ CD133⁺ CD90⁺, and the MPPs are CD34⁺ CD38⁻ CD45RA⁻ CD133⁺ CD90⁻.

### Notch Ligands

An agonist of Notch function, also referred to as Notch agonist or Notch ligand, is an agent that promotes, i.e., causes or increases, activation of Notch pathway function. As used herein, “Notch function” means a function mediated by the Notch signaling (signal transduction) pathway, including but not limited to nuclear translocation of the intracellular domain of Notch, nuclear translocation of RBP-JK or its Drosophila homolog Suppressor of Hairless; activation of bHLH genes of the Enhancer of Split complex, e.g., Mastermind; activation of the HES-1 gene or the KBF2 (also called CBF1) gene; inhibition of Drosophila neuroblast segregation; and binding of Notch to Delta, Jagged/Serrate, Fringe, Deltex or RBP-Jκ/Suppressor of Hairless, or homologs or analogs thereof. See generally the review article by Kopan et al., Cell 137:216-233, 2009, for a discussion of the Notch signal transduction pathway and its effects upon activation; see also Jarriault et al., Mol. Cell. Biol. 18:7423-7431, 1998.

Exemplary Notch agonists are the extracellular binding ligands Delta and Serrate which bind to the extracellular domain of Notch and activate Notch signal transduction, or a fragment of Delta or Serrate that binds to the extracellular domain of Notch and activates Notch signal transduction. Nucleic acid and amino acid sequences of Delta and Serrate have been isolated from several species, including human, are known in the art, and are disclosed in International Patent Publication Nos. WO 93/12141, WO 96/27610, WO 97/01571, and Gray et al., Am. J. Path. 154:785-794, 1999.

In a preferred embodiment, the Notch agonist is an active fragment of a Delta or Serrate protein consisting of the extracellular domain of the protein fused to a myc epitope tag (Delta^{ext-myc} or Serrate^{ext-myc}, respectively) or an active fragment of a Delta or Serrate protein consisting of the extracellular domain of the protein fused to the Fc portion of IgG (Delta^{ext-IgG} or Serrate^{ext-IgG} respectively). Notch agonists include but are not limited to Notch proteins and analogs and derivatives (including fragments) thereof; proteins that are other elements of the Notch pathway and analogs and derivatives (including fragments) thereof, antibodies thereto and fragments or other derivatives of such antibodies containing the binding region thereof, nucleic acids encoding the proteins and derivatives or analogs; as well as proteins and derivatives and analogs thereof which bind to or otherwise interact with Notch proteins or other proteins in the Notch pathway such that Notch pathway activity is promoted. Such agonists include, but are not limited to, Notch proteins and derivatives thereof comprising the intracellular domain, Notch nucleic acids encoding the foregoing, and proteins comprising the Notch-interacting domain of Notch ligands (e.g., the extracellular domain of Delta or Serrate). Other agonists include but are not limited to RBPJκ/Suppressor of Hairless or Deltex. Fringe can be used to enhance Notch activity, for example in conjunction with Delta protein. These proteins, fragments and derivatives thereof can be recombinantly expressed and isolated or can be chemically synthesized.

An agonist of Notch also can be a peptidomimetic or peptide analog or organic molecule that binds to a member of the Notch signaling pathway. Such an agonist can be identified by binding assays selected from those known in the art, for example the cell aggregation assays described in Rebay et al., Cell 67:687-699, 1991 and in International Patent Publication No. WO 92/19734 (both incorporated herein by reference in their entirety).

A Notch agonist is a protein consisting of at least a fragment of a protein encoded by a Notch-interacting gene which mediates binding to a Notch protein or a fragment of Notch, which fragment of Notch contains the region of Notch responsible for binding to the agonist protein, e.g., epidermal growth factor-like repeats 11 and 12 of Notch. Notch interacting genes, as used herein, shall mean the genes Notch, Delta, Serrate, RBPJκ, Suppressor of Hairless and Deltex, as well as other members of the Delta/Serrate family or Deltex family which may be identified by virtue of sequence homology or genetic interaction and more generally, members of the “Notch cascade” or the “Notch group” of genes, which are identified by molecular interactions (e.g., binding in vitro, or genetic interactions (as depicted phenotypically, e.g., in Drosophila). Exemplary fragments of Notch-binding proteins containing the region responsible for binding to Notch are described in U.S. Pat. Nos. 5,648,464; 5,849,869; and 5,856,441 (all incorporated herein by reference in their entirety). U.S. Pat. No. 5,780,300 to Artavanis-Tsakonas et al. (incorporated herein by reference), describes Notch agonists include reagents that promote or activate cellular processes that mediate the maturation or processing steps required for the activation of Notch or a member of the Notch signaling pathway, such as the furin-like convertase required for Notch processing, Kuzbanian, the metalloprotease-disintegrin (ADAM) thought to be required for the activation of the Notch pathway upstream or parallel to Notch (Schlondorff and Blobel, J. Cell Sci. 112:3603-3617, 1999), or, more generally, cellular trafficking and processing proteins such as the rab family of GTPases required for movement between cellular compartments (for a review on Rab GTPases, see Olkkonen and Stenmark, Int. Rev. Cytol. 176:1-85, 1997). The agonist can be any molecule that increases the activity of one of the above processes, such as a nucleic acid encoding a furin, Kuzbanian or rab protein, or a fragment or derivative or dominant active mutant thereof, or a peptidomimetic or peptide analog or organic molecule that binds to and activates the function of the above proteins.

U.S. Pat. No. 5,780,300 further discloses classes of Notch agonist molecules (and methods of their identification) which can be used to activate the Notch pathway, for example molecules that trigger the dissociation of the Notch ankyrin repeats with RBP-JK, thereby promoting the translocation of RBP-JK from the cytoplasm to the nucleus.

In some embodiments, DXI is used. The Notch agonist DXI is an immobilized fragment of a Delta1 consisting of the extracellular domain of the protein fused to the Fc portion of IgG (Delta1^{ext-IgG} or DXI), as described in U.S. Pat. No. 7,399,633 (incorporated herein by reference) or an immobilized Notch-1 or Notch-2 specific antibody, as described in U.S. Pat. No. 10,208,286 (incorporated herein by reference). Preferably, Delta1^{ext-IgG} is immobilized on the surface of the cell culture dishes.

Release of Expanded HSPCs from the Hydrogel

The cultured HSPCs can be released from the hydrogel (e.g., an alginate/poly-lysine hydrogel or an alginate hydrogel) which can be depolymerized in the presence of chelating agent. For example, a chelating agent, such as sodium citrate or EDTA, can be used to induce depolymerization of the hydrogel.

### Genetically Engineered HSPCs

The HSPCs can be genetically engineered to express a molecule or molecules of interest, such as a protein, nucleic acid or carbohydrate before, during or after the culturing step. The HSPCs may be genetically engineered to reduce or eliminate expression a molecule or molecules of interest, such as a protein, nucleic acid or carbohydrate. The HSPCs can be genetically engineered before, during or after the culturing step.

In some embodiments, the HSPCs are genetically engineered to express a protein of interest, such as a protein, polypeptide or peptide (collectively referred to as a protein). In some embodiments, the HSPCs are genetically engineered to express a normal (e.g., wild type) version of a gene. In some embodiments, the HSPCs are genetically engineered to express an altered version of an endogenous gene (e.g., a gene encoding a mutant protein).

In some embodiments, the HSPCs are genetically engineered to express a heterologous protein. As used herein, a heterologous protein is a protein not normally expressed by the HSPCs. As used herein, a heterologous protein is a protein not normally expressed by the cells derived from the HSPCs. In some embodiments, the heterologous protein is an antigen recognizing receptor.

An HSPC population can be genetically modified before or during or after culturing. In some embodiments, the HSPCs are genetically engineered during culturing. In some embodiments, the HSPCs are genetically engineered after culturing. In some embodiments, the HSPCs are genetically engineered before culturing. In some embodiments, the HSPCs are genetically engineered during or after enrichment. In some embodiments, the HSPCs are genetically modified after enrichment.

As one example, a genetic modification can be selected to provide a therapeutic benefit against a condition that is inherited. In some embodiments, the condition can be Grave's Disease, rheumatoid arthritis (RA), pernicious anemia, Multiple Sclerosis (MS), inflammatory bowel disease (IBD), systemic lupus erythematosus (SLE), adenosine deaminase deficiency (ADA-SCID) or severe combined immunodeficiency disease (SCID), Wiskott-Aldrich syndrome (WAS), chronic granulomatous disease (CGD), Fanconi anemia (FA), Battens disease, adrenoleukodystrophy (ALD) or metachromatic leukodystrophy (MLD), muscular dystrophy (MD), pulmonary aveolar proteinosis (PAP), pyruvate kinase deficiency, Shwachmann-Diamond-Blackfan anemia, dyskeratosis congenita, cystic fibrosis (CF), Parkinson's disease, Alzheimer's disease, or amyotrophic lateral sclerosis (Lou Gehrig's disease). In some embodiments, depending on the condition, the genetic modification can be the introduction of a therapeutic gene that encodes a protein and/or a gene whose function has been interrupted. Exemplary therapeutic gene and gene products include: soluble CD40; CTLA; Fas L; antibodies to CD4, CD5, CD7, CD52, and the like; antibodies to IL-1, IL-2, IL-6; IL-4; IL-10; IL-12; IL-13; IL-1Ralpha, sIL-1RI, sIL-1RII; sTNFR-I; sTNFR-II; antibodies to TNF; p53, PTPN22, and DRB1*1501/DQB1*0602; globin family genes; WAS; phox; FANC family genes; dystrophin; pyruvate kinase; CLN3; ABCD1; arylsulfatase A; SFTPB; SFTPC; NLX2.1; ABCA3; GATA1; ribosomal protein genes; TERT; TERC; DKC1; TINF2; CFTR; LRRK2; PARK2; PARK7; PINK1; SNCA; PSEN1; PSEN2; APP; SOD1; TDP43; FUS; ubiquilin 2; and/or C90RF72.

As another example, a genetic modification can be introduction of a therapeutic gene selected to provide a therapeutically effective response against diseases related to red blood cells and clotting. In some embodiments, the disease is a hemoglobinopathy like thalassemia, or a sickle cell disease/trait. The therapeutic gene can be, for example, a gene that induces or increases production of hemoglobin; induces or increases production of beta-globin, or alpha-globin; or increases the availability of oxygen to cells in the body. The therapeutic gene can be, for example, HBB or CYB5R3. Exemplary effective treatments can, for example, increase blood cell counts, improve blood cell function, or increase oxygenation of cells in patients. In another particular embodiment, the disease is hemophilia. The therapeutic gene can be, for example, a gene that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary therapeutic genes include F8 and F9. Exemplary effective treatments can, for example, increase or induce the production of coagulation/clotting factors VIII and IX; improve the functioning of coagulation/clotting factors VIII and IX, or reduce clotting time in subjects.

As another example, the genetic modification can be introduction of a therapeutic gene to provide a therapeutically effective response against a lysosomal storage disorder. In particular embodiments, the lysosomal storage disorder is mucopolysaccharidosis (MPS) type I; MPS type II, or Hunter Syndrome; MPS III or Sanfilippo syndrome; MPS IV or Morquio syndrome; MPS V; MPS VI or Maroteaux-Lamy syndrome; MPS VII or sly syndrome; alpha-mannsidosis; beta-mannosidosis; glycogen storage disease type I, also known as GSDI, von Gierke disease, or Tay Sachs; Pompe disease; Gaucher disease; Fabry disease. The therapeutic gene can be, for example, a gene encoding or inducing production of an enzyme, or that otherwise causes the degradation of mucopolysaccharides in lysosomes. Exemplary therapeutic genes include IDUA or iduronidase, IDS, GNS, HGSNAT, SGSH, NAGLU, GUSB, GALNS, GLBJ, ARSB, and HYALL. Exemplary effective genetic therapies for lysosomal storage disorders may, for example, encode or induce the production of enzymes responsible for the degradation of various substances in lysosomes; reduce, eliminate, prevent, or delay the swelling in various organs, including the head (exp. Macrosephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a genetic modification can be introduction of a therapeutic gene to provide a therapeutically effective response against a hyperproliferative disease. In particular embodiments the hyperproliferative disease is cancer. The therapeutic gene can be, for example, a tumor suppressor gene, a gene that induces apoptosis, a gene encoding an enzyme, a gene encoding an antibody, or a gene encoding a hormone. Exemplary therapeutic genes and gene products include 101F6, 123F2 (RASSF1), 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CFTR, CNTF, COX-1, CSFIR, CTS-1, cytosine deaminase, DBCCR-1, DCC, Dp, DPC-4, ElA, E2F, EBRB2, erb, ERBA, ERBB, ETS1, ETS2, ETV6, Fab, FCC, FGF, FGR, FHIT, fins, FOX, FUS 1, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-4, IL-5, IL-6, IL-7, IL-8, IL-9, IL-10, IL-11 IL-12, ING1, interferon alpha, interferon beta, interferon gamma, IRF-1, JUN, KRAS, LCK, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p53, p57, p73, p300, PGS, PIM1, PL6, PML, PTEN, raf, RaplA, ras, Rb, RB1, RET, rks-3, ScFv, scFV ras, SEM A3, SRC, TALI, TCL3, TFPI, thrombospondin, thymidine kinase, TNF, TP53, trk, T-VEC, VEGF, VHL, WT1, WT-1, YES, and zac1. Exemplary effective genetic therapies can suppress or eliminate tumors, result in a decreased number of cancer cells, reduced tumor size, slow or eliminate tumor growth, or alleviate symptoms caused by tumors, and the like.

As another example, a genetic modification can be introduction of a therapeutic gene selected to provide a therapeutically effective response against an infectious disease. In particular embodiments the infectious disease is human immunodeficiency virus (HIV). The therapeutic gene can be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include alpha2beta1; alphavbeta3; alphavbeta5; alphavbeta6; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; alpha-dystroglycan; LDLR/alpha2MR/LRP; PVR; PRR1/HveC; and laminin receptor. A therapeutically effective amount for the treatment of HIV, for example, can increase the immunity of a subject against HIV, ameliorate a symptom associated with AIDS or HIV, or induce an innate or adaptive immune response in a subject against HIV. An immune response against HIV can include antibody production and result in the prevention of AIDS and/or ameliorate a symptom of AIDS or HIV infection of the subject, or decrease or eliminate HIV infectivity and/or virulence.

In some embodiments, the HSPCs can be genetically engineered to express a heterologous antigen recognizing receptor(s) that binds to an antigen of interest. In certain embodiments, the antigen recognizing receptor is a chimeric antigen receptor (CAR). In certain embodiments, the antigen recognizing receptor is a T-cell receptor (TCR). The antigen recognizing receptor can bind to a tumor antigen or a pathogen antigen.

In some embodiments, the antigen recognizing receptor binds to a tumor antigen, such as carbonic anhydrase IX (CA1X), carcinoembryonic antigen (CEA), CD8, CD7, CD10, CD19, CD20, CD22, CD30, CD33, CLL1, CD34, CD38, CD41, CD44, CD49c, CD49f, CD56, CD66c, CD73, CD74, CD104, CD133, CD138, CD123, CD142, CD44V6, an antigen of a cytomegalovirus (CMV) infected cell (e.g., a cell surface antigen), cutaneous lymphocyte-associated antigen (CLA; a specialized glycoform of P-selectin glycoprotein ligand-1 (PSGL-1)), epithelial glycoprotein-2 (EGP-2), epithelial glycoprotein-40 (EGP-40), epithelial cell adhesion molecule (EpCAM), receptor tyrosine-protein kinases erb-B 2,3,4 (erb-B2,3,4), folate-binding protein (FBP), fetal acetylcholine receptor (AChR), folate receptor-alpha, Ganglioside G2 (GD2), Ganglioside G3 (GD3), human Epidermal Growth Factor Receptor 2 (HER2), human telomerase reverse transcriptase (hTERT), Interleukin-13 receptor subunit alpha-2 (IL-13R-alpha2), kappa-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PRI), Tyrosinase, Survivin, hTERT, EphA2, an NKG2D ligand, cancer-testis antigen NY-ESO-1, oncofetal antigen (h5T4), prostate stem cell antigen (PSCA), prostate-specific membrane antigen (PSMA), ROR1, tetraspanin 8 (TSPAN8), tumor-associated glycoprotein 72 (TAG-72), vascular endothelial growth factor R2 (VEGF-R2), Wilms tumor protein (WT-1), cytokine receptor-like factor 2 (CRLF2), BCMA, GPC3, NKCS1, EGF1R, EGFR-VIII, or ERBB.

The HSPCs can be genetically modified by any suitable technique. Methods of preparing genetically modified HSPCs generally include introducing into the HSPCs a polynucleotide(s). The polynucleotide(s) encoding the desired molecule can be introduced into the HSPCs, before, during or after culturing. Desired polynucleotides containing genes can be introduced into the HSPCs by any suitable method known in the art, including transfection, electroporation, microinjection, lipofection, calcium phosphate mediated transfection, infection with a viral or bacteriophage vector containing the gene sequences (e.g., a retrovirus, such as a lentivirus), cell fusion, chromosome-mediated gene transfer, microcell-mediated gene transfer, spheroplast fusion, using CRISPR or other rare-cutting endonuclease (e.g., TALE-nuclease or Cas9 endonuclease), zinc finger technology, and the like. Numerous techniques are known in the art for the introduction of foreign genes into cells (see e.g., Loeffler and Behr, Meth. Enzymol. 217:599-618, 1993; Cohen et al., Meth. Enzymol. 217:618-644, 1993; Cline, Pharmac. Ther. 29:69-92, 1985 (all incorporated herein by reference in their entirety), and can be used, provided that the necessary physiological functions of the recipient cells are not disrupted. The technique should provide for the stable transfer of the gene to the cell, so that the gene, if desired, is expressible by the cell and preferably heritable and expressible by its cell progeny. In some embodiments, the method of transfer includes the transfer of a selectable marker or tag sequence to the cells. The cells are then placed under selection to isolate those cells that have taken up and are expressing the transferred gene. In a preferred embodiment, the polynucleotide(s) or genes are included in lentiviral vectors in view of being stably expressed in the cells.

### Cryopreservation of the HSPCs

An HSPC population can be cryopreserved. Typically, the HSPCs are released after removal from encapsulation. The terms “frozen/freezing” and “cryopreserved/cryopreserving” are used interchangeably in the present application. Cryopreservation can be any method known in the art that preserves cells in viable form. The freezing of cells is ordinarily destructive. On cooling, water within the cell freezes. Injury then occurs by osmotic effects on the cell membrane, cell dehydration, solute concentration, and ice crystal formation. As ice forms outside the cell, available water is removed from solution and withdrawn from the cell, causing osmotic dehydration and raised solute concentration which eventually destroys the cell. For a discussion, see Mazur, Cryobiology 14:251-272, 1977.

These injurious effects can be circumvented by (a) use of a cryoprotective agent, (b) control of the freezing rate, and (c) storage at a temperature sufficiently low to minimize degradative reactions.

Cryoprotective agents which can be used include but are not limited to dimethyl sulfoxide (DMSO) (Lovelock and Bishop, Nature 183:1394-1395, 1959; Ashwood-Smith, Nature 190:1204-1205. 1961), glycerol, polyvinyl/pyrrolidine (Rinfret, Ann. N.Y. Acad. Sci. 85:576, 1960), polyethylene glycol (Sloviter and Ravdin, Nature 196:548, 1962), albumin, dextran, sucrose, ethylene glycol, i-erythritol, D-ribitol, D-mannitol (Rowe et al., Fed. Proc. 21:157, 1962), D-sorbitol, i-inositol, D-lactose, choline chloride (Bender et al., J. Appl. Physiol. 15:520, 1960), amino acids (Phan The Tran and Bender, Exp. Cell Res. 20:651, 1960), methanol, acetamide, glycerol monoacetate (Lovelock, Biochem. J. 56:265, 1954), inorganic salts (Phan The Tran and Bender, Proc. Soc. Exp. Biol. Med. 104:388, 1960; Phan The Tran and Bender, in Radiobiology, Proceedings of the Third Australian Conference on Radiobiology, Ilbery ed., Butterworth, London, p. 59, 1961), and CryoStor™ CS5 or CS10 (BioLife Solutions Inc., Bothell, Wash.). In a preferred embodiment, DMSO is used, a liquid which is nontoxic to cells in low concentration. In some embodiments, addition of plasma (e.g., to a concentration of 20-25%) can augment the protective effect of DMSO. In some embodiments, addition of human serum albumin (e.g., to a concentration of 2-10%) can augment the protective effect of DMSO. After addition of DMSO, cells should be kept at 0° C. until freezing, since DMSO concentrations of about 1% are toxic at temperatures above 4° C.

Another example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 10 per minute and stored in the vapor phase of a liquid nitrogen storage tank.

A controlled slow cooling rate can be important. Different cryoprotective agents (Rapatz et al., Cryobiology 5(1):18-25, 1968) and different cell types have different optimal cooling rates (see e.g., Rowe and Rinfret, Blood 20:636, 1962; Rowe, Cryobiology 3(1):12-18, 1966; Lewis, et al., Transfusion 7(1):17-32, 1967; and Mazur, Science 168:939-949, 1970 for effects of cooling velocity on survival of marrow-stem cells and on their transplantation potential). The heat of fusion phase where water turns to ice should be minimal. The cooling procedure can be carried out by use of, e.g., a programmable freezing device or a methanol bath procedure.

Programmable freezing apparatuses allow determination of optimal cooling rates and facilitate standard reproducible cooling. Programmable controlled-rate freezers such as Cryomed or Planar permit tuning of the freezing regimen to the desired cooling rate curve. For example, for marrow cells in 10% DMSO and 20% plasma, the optimal rate is 1° to 3° C./minute from 0° C. to −80° C. In a preferred embodiment, this cooling rate can be used. The container holding the cells must be stable at cryogenic temperatures and allow for rapid heat transfer for effective control of both freezing and thawing. Sealed plastic vials (e.g., Nunc, Wheaton™ Cryule™ ampules) or glass ampules can be used for multiple small amounts (1-2 ml) or larger amounts (e.g., 5 to 30 ml), while larger volumes (20-200 ml) can be frozen in polyolefin bags or ethylene vinyl acetate freezer bags (e.g., OriGen) held between metal plates for better heat transfer during cooling. By way of example, bags of bone marrow cells have been successfully frozen by placing them in −80° C. freezers which, fortuitously, gives a cooling rate of approximately 3° C./minute).

In an alternative embodiment, the methanol bath method of cooling can be used. The methanol bath method is well-suited to routine cryopreservation of multiple small items on a large scale. The method does not require manual control of the freezing rate nor a recorder to monitor the rate. In a preferred embodiment, DMSO-treated cells are pre-cooled on ice and transferred to a tray containing chilled methanol which is placed, in turn, in a mechanical refrigerator (e.g., Harris or Revco) at −80° C. Thermocouple measurements of the methanol bath and the samples indicate the desired cooling rate of 1° to 3° C./minute. After at least two hours, the specimens have reached a temperature of −80° C. and can be placed directly into liquid nitrogen (−196° C.) for permanent storage.

After thorough freezing, an HSPC population can be rapidly transferred to a long-term cryogenic storage vessel. In a preferred embodiment, samples can be cryogenically stored in liquid nitrogen (−196° C.) or its vapor (−165° C.). In another preferred embodiment, samples can be cryogenically stored in liquid nitrogen vapor phase (e.g., −130° C.). Such storage is greatly facilitated by the availability of highly efficient liquid nitrogen refrigerators, which resemble large Thermos containers with an extremely low vacuum and internal super insulation, such that heat leakage and nitrogen losses are kept to an absolute minimum.

Suitable racking systems are commercially available and can be used for cataloguing, storage, and retrieval of individual specimens.

Other methods of cryopreservation of viable cells, or modifications thereof, are available and envisioned for use (e.g., cold metal-mirror techniques; Livesey and Linner, Nature 327:255, 1987; Linner et al., J. Histochem. Cytochem. 34(9):1123-1135, 1986; see also U.S. Pat. No. 4,199,022 by Senkan et al., U.S. Pat. No. 3,753,357 by Schwartz, U.S. Pat. No. 4,559,298 by Fahy).

Cryopreserved or frozen cells are preferably thawed quickly (e.g., in a water bath maintained at 37°-41° C.) and chilled immediately upon thawing. In a specific embodiment, the vial containing the frozen cells can be immersed up to its neck in a warm water bath; gentle rotation will ensure mixing of the cell suspension as it thaws and increase heat transfer from the warm water to the internal ice mass. As soon as the ice has completely melted, the vial can be immediately placed in ice.

In an embodiment, a cryopreserved HSPC population is thawed, or a portion thereof, can be infused into a human patient in need thereof or used to generate other cells types. Several procedures, relating to processing of the thawed cells, are available and can be employed if deemed desirable.

It can be desirable to treat the cells to prevent cellular clumping upon thawing. To prevent clumping, various procedures can be used, including but not limited to, the addition of DNase before and/or after freezing (Spitzer et al., Cancer 45:3075-3085, 1980), low molecular weight dextran and citrate, hydroxyethyl starch (Stiff et al., Cryobiology 20:17-24, 1983), and the like.

The cryoprotective agent, if toxic in humans, should be removed prior to therapeutic use of the thawed HSPC population. In an embodiment employing DMSO as the cryopreservative, it is preferable to omit this step, in order to avoid cell loss. However, where removal of the cryoprotective agent is desired, the removal is preferably accomplished upon thawing.

One way in which to remove the cryoprotective agent is by dilution to an insignificant concentration. This can be accomplished by addition of medium, followed by, if necessary, one or more cycles of centrifugation to pellet cells, removal of the supernatant, and resuspension of the cells. For example, intracellular DMSO in the thawed cells can be reduced to a level (less than 1%) that will not adversely affect the recovered cells. This is preferably done slowly to minimize potentially damaging osmotic gradients that occur during DMSO removal.

After removal of the cryoprotective agent, cell count (e.g., by use of a hemocytometer) and viability testing (e.g., by trypan blue exclusion; Kuchler, in Biochemical Methods in Cell Culture and Virology, Dowden, Hutchinson & Ross, Stroudsburg, Pa., pp. 18-19, 1977; Methods in Medical Research, Eisen et al., eds., Vol. 10, Year Book Medical Publishers, Inc., Chicago, pp. 39-47, 1964) can be done to confirm cell survival. The percentage of viable antigen (e.g., CD56) positive cells can be determined by calculating the number of antigen positive cells that exclude 7-AAD (or other suitable dye excluded by viable cells) in an aliquot of the cells, divided by the total number of nucleated cells (TNC) (both viable and non-viable) in the aliquot of the cells. The number of viable antigen positive cells can be then determined by multiplying the percentage of viable antigen positive cells by the TNC.

Prior to cryopreservation and/or after thawing, the total number of nucleated cells, or in a specific embodiment, the total number of CD34⁺ and/or CD133⁺ cells can be determined. For example, total nucleated cell count can be performed by using a hemocytometer and exclusion of trypan blue dye. Specimens that are of high cellularity can be diluted to a concentration range appropriate for manual counting. Final cell counts for products are corrected for any dilution factors. Total nucleated cell count=viable nucleated cells per mL×volume of product in ml. The number of CD34⁺ positive cells in the sample can be determined, e.g., by the use of flow cytometry using anti-CD34 monoclonal antibodies conjugated to a fluorochrome. Similarly, the number of CD133⁺ positive cells in the sample can be determined, e.g., by the use of flow cytometry using anti-CD133 monoclonal antibodies conjugated to a fluorochrome.

### EXAMPLES

### Example 1: Generation of an Encapsulated HSPC Population

The following example describes the preparation of an encapsulated HSPC Population.
Equipment required: Buchi encapsulator B-395 Pro.
Materials required:
a. Na-alginate solution. 1 to approximately 3% (w/v) Na-alginate (Novamatrix) sterile filtered solution pH 7.0-7.4 at room temperature. The Na-alginate was mixed with the cells and formed into Ca-alginate beads encapsulating the cells.
b1. Hardening (gelling) solution for an alginate/poly-lysine (ALG/PLL) hydrogel: 10 mM MOPS (Morpholinopropanesulfomic acid, Fisher); 100 mM CaCl₂ (dihydrate, Sigma); 0.01% to approximately 0.1% poly-L-lysine (PLL, Sigma), pH 7.4 at room temperature.
b2. Hardening (gelling) solution for an alginate (ALG) hydrogel: 10 mM MOPS; 100 mM CaCl₂ (dihydrate, Sigma); pH 7.4 at room temperature.
c. 1% to approximately 3% Na-alginate solution. 2 mL of 1.8% Na-alginate solution in 118 mL of MOPS buffer. Gelling solution reacts with the Na-alginate to form Ca/PLL-alginate double network beads.
d. Ca-solubilization solution. 50 mM Sodium citrate (Sigma); 0.45% (w/v) NaCl or 50 mM EDTA (Invitrogen) NaCl; 10 mM MOPS pH 7.4 at room temperature. The solution solubilizes the Ca-alginate network within the bead to release unbalanced Alginate.
e. MOPS (washing) buffer: 10 mM MOPS 0.85% (w/v) NaCl (Fisher). pH adjusted to 7.4 at room temperature. (The MOPS washing step is optional and used to wash the capsules after each production step to remove unreacted components. The buffer also helps to maintain physiological conditions.)
f. Required Volumes: The following volumes were required when producing 12 mL of alginate-PLL microcapsules containing human cells at a concentration of 10⁴-10⁷ cells/mL. Note: All non-sterile solutions were passed (filtered) through a 0.2 μm liquid filter membrane when being delivered to the enclosed reaction vessel during capsule production to maintain sterile conditions.

1. 12 mL 1.8% sterile alginate solution.

2. 250 mL Hardening solution (non-sterile)

3. 1000 mL MOPS washing buffer (non-sterile)

4. 200 mL solubilization solution (non-sterile)

g. Enriched CD34⁺ HSPCs were prepared from pooled cord blood units as described in U.S. Patent Application Publication No. 2013/0095079 (incorporated herein by reference). Generally, CD34⁺ cells from at least 2 or 4 cord blood units were pooled, either before or after enrichment for CD34⁺ cells.

### Exemplary Steps:

The preparation of an alginate-poly-L-lysine (ALG/PLL) hydrogel is described below. An alginate hydrogel without a poly-lysine can be prepared as described below, using hardening solution b2 instead of b1 as described above.

1. A container (e.g., a 2 L glass vessel) was prepared with the 750 μm nozzle in place and spray with 70% alcohol as described below.
2. All solutions and labware were prepared.
3. 200 mL of the hardening solution (b1) was pumped into the autoclaved reaction vessel using the pressure bottle. The hardening solution was sterilized by pumping it through the autoclaved liquid filter before entering the reaction vessel.
4. The reaction vessel was placed under a laminar air flow cabinet.
5. A culture of cells at a concentration of 1×10⁴-1×10⁷ cells/mL was prepared under a laminar flow cabinet. The cells were centrifuged, the supernatant decanted and the pellet was re-suspended in 2 mL sterile MOPS washing buffer. 10 mL of the 1.8% sodium-alginate solution was added to the re-suspended cells using a sterile 25 mL pipette. The solution was mixed by re-aspirating it into the 25 ml pipette twice. It was confirmed that no or few air bubbles were introduced during resuspension and mixing. A sterile 20 ml syringe was attached to an autoclaved 15 cm long silicone tube (inner diameter 3 mm). This tube was used to transport the cells into the syringe, after which the tube was removed. The syringe was filled with the cell-alginate suspension and the syringe was attached to the bead producing unit on the reaction vessel. The reaction vessel was fixed to the encapsulator control unit, which was placed on a bench (not in the laminar flow cabinet).
6. The bypass cup was placed directly underneath the nozzle to prevent any unwanted alginate landing in the gelling solution.
7. The magnetic stirrer was turned on.
8. Bead formation was started with the following parameters: nozzle size: 750 μm; stir rate: 30%; vibration frequency: 300 Hz, voltage 1000 V; and syringe pump speed 35 mL/min. Once a stable droplet chain of alginate droplets has been established the bypass cup was removed from underneath the nozzle and the droplets were allowed to land in the stirred hardening solution. (Note: Just before all the alginate solution is pumped through the syringe, the bypass cup was placed underneath the nozzle once more).
9. The droplets were allowed to harden for 10 minutes in the hardening solution bath to form the Ca/PLL-alginate and Ca/ALG beads and then the gelling solution was drained. Note: The beads and later the capsules should always be covered by a small amount of liquid to prevent clumping; otherwise resuspension of the beads and capsules can be difficult, and the membrane could be damaged.
10. 200 mL of MOPS buffer was pumped in, stirred for 1 min and then the buffer was drained off. The washing step was repeated once.
11. 150 mL Ca-solubilization solution was pumped in and stirred approximately 10 min to dissolve the alginate/Ca₂⁺ network. The appropriate dissolution time was dependent on the molecular weight and purity of the alginate, and the susceptibility of the encapsulated cells to the depolymerization solution. (No solubilization process is needed for ALG group alone.)
12. The depolymerization solution was drained off.
13. 150 mL MOPS washing buffer was pumped in, the capsules were resuspended and transferred into the bead collecting flask.
14. The washing buffer was replaced with the culture medium.
15. The capsules were transferred aseptically into a flask or bioreactor to allow the cells to expand.

### Example 2: Generation of an Encapsulated HSPC Population

The following example describes the preparation of an encapsulated HSPC Population.

Equipment required: Buchi encapsulator B-395 Pro.
Materials required:
a. Alginate (ALG) Gelling Solution: 1.5% Alginate-UP LVG was prepared in SFEM II media supplemented with 50 ng/mL SCF, TPO, Flt3-L, IL-6 and 10 ng/mL IL-3 and filtered using a 0.2 μm bottle top filter. A volume of 30 mL was prepared for this experiment and stored at 4-8° C. till the time of use.
b. Calcium chloride hardening solution: 2.5% CaCl₂) solution was prepared using Milli Q water and filtered using a 0.2 μm bottle top filter. A volume of 500 mL was prepared and stored at room temperature till the time of use
c. Calcium chloride+poly-L-lysine hardening solution: 2.5% CaCl₂) and 0.01% poly-L-lysine solution was prepared in Milli Q water and filtered using a 0.2 μm bottle top filter. A volume of 500 ml was prepared and stored at 4-8° C. till the time of use.
d. 150 mM NaCl solution: 0.85 g of NaCl was dissolved in Milli Q water and filtered using a 0.2 μm bottle top filter. A volume of 1 L was prepared stored at room temperature till the time of use.
e. 50 mM EDTA solution: 100 mL of 0.5M EDTA solution and 0.43 g of NaCl was diluted to a final volume of 1 L using Milli Q water. The solution was filtered using a 0.2 μm bottle top filter and stored at 4-8° C. till the time of use.
f. Dissolving solution: 100 mL of 0.5 M EDTA and 0.85 g of NaCl was diluted to a final volume of 1 L using SFEM II media supplemented with 50 ng/mL SCF, TPO, Flt3-L, IL-6 and 10 ng/mL IL-3. The solution was filtered using a 0.2 μm bottle top filter and stored at 4-8° C. till the time of use.
g. Enriched CD34⁺ HSPCs are prepared from pooled cord blood units as described in US Patent Application Publication No. 2013/0095079 (incorporated herein by reference). Generally, CD34⁺ cells from at least 2 or 4 cord blood units were pooled, either before or after enrichment for CD34⁺ cells.

### Exemplary Steps:

CD34⁺ HSPCs enriched from pooled cord blood units were resuspended in SFEM II media supplemented with 50 ng/ml rhSCF, rhTPO, rhFlt3-L, rhIL-6 and 10 ng/mL rhIL-3 and cultured overnight in an incubator set to 37° C., 5% CO₂ and ambient 02.

To prepare for encapsulation, cells cultured overnight were harvested and then re-suspended in 1.5% alginate gelling solution at a density of 5×10⁵ cells/mL. The BUCHI encapsulator was fitted with a 750 μm nozzle and set up using the settings described below.

| Cell Seeding | Vibration | | | Syringe Pump |
|---|---|---|---|---|
| Density | Frequency | Stir Rate | Voltage | Speed |
| 5 × 10⁵ | 300 Hz | 30% | 1000 V | 35 mL/min |
| cells/mL | | | | |

200 mL of either calcium chloride hardening solution or calcium chloride+poly-L-lysine hardening solution was added to the capture vessel below the encapsulator. These solutions respectively crosslink with ALG to encapsulate cells in ALG or ALG+PLL microcapsules respectively. The cell suspension in alginate solution was pulled into a syringe and fitted to the bead producing unit. The syringe pump was started, and the beads were collected in the vessel containing the hardening solution over 10 minutes. The hardening solution was then drained from the vessel by opening the draining valve leaving the capsules with encapsulated cells within. ALG microcapsules were then washed in 0.85% NaCl solution for 5 minutes while ALG+PLL microcapsules were washed in 150 mL of EDTA solution to dissolve the CaCl₂)/PLL network. After aspirating the washing solution, the capsules were transferred to a sterile bottle in 200 mL PBS solution for a few minutes. Following this, the PBS solution was aspirated, and the capsules were re-suspended in expansion media for culture.

### Example 3: Culture of Encapsulated HSPCs

Culture medium consisted of StemSpan Serum Free Expansion Medium II (SFEM II; StemCell Technologies) supplemented with 50 ng/mL each recombinant human SCF (Miltenyi Biotec), rhFlt3-Ligand (Miltenyi Biotec), rhTPO (Miltenyi Biotec), and rhIL-6 (Miltenyi Biotec), and 10 ng/ml rhIL-3 (Miltenyi Biotec).

Cells encapsulated in either ALG or ALG+PLL microcapsules were cultured in culture medium in either static culture vessels such as T-flasks or in roller bottles with rotations set to 2 rpm (dynamic culture). Cultures were maintained for 14 days with a complete media exchange performed at least once during the culture duration.

Following culture time course, the microcapsules were transferred into suitable sterile centrifuge tubes and allowed to settle. The culture media was then aspirated, and residual capsules were washed in PBS solution. The capsules were then resuspended in dissolving solution and placed on a rotator for 10 minutes to release the cells from the microcapsules. The cell mixture was then strained through a 70 μm cell strainer to remove alginate capsule debris. The cells in the elute were then centrifuged and resuspended in a desired media or buffer.

### Example 4: Comparison of 2D and 3D Culture Systems

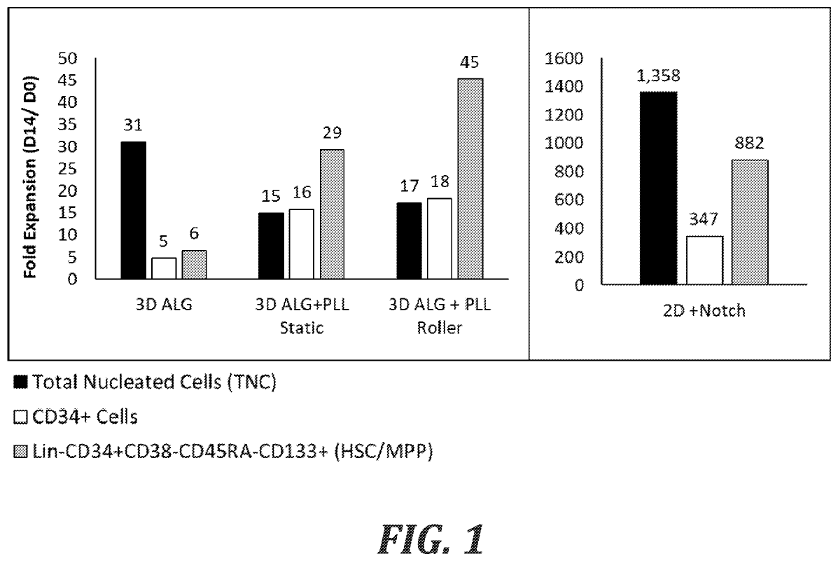

For the expansion of cord blood HSPCs, during a 14-day culture with SFEMII medium supplemented with 5 growth factors (SCF, Flt3-L, TPO, IL-3 and IL-6), a significant expansion was observed in the 2D culture system (using the Notch ligand DXI). In comparison, an expansion of CD34⁺ cells and HSCs was observed in three versions of the 3D culture, alginate alone (ALG), ALG/PLL in a static format (in tissue culture treated T flasks (e.g., Corning cell culture flasks T-25 and T-75 cell culture flasks) and ALG/PLL in 500 mL roller bottles (CELLTREAT). As shown in FIG. 1, this observation was applicable to total nucleated cells (TNC), CD34⁺ populations and HSC and MPP populations (Lin⁻ CD34⁺ CD38⁻ CD45RA⁻ CD133⁺ CD90⁺/− cells).

As shown in Table 1, a significant loss of the CD34⁺ population was observed in the 2D culture system (using a Notch ligand, DXI). (The 2D culture system is generally performed as described in U.S. Patent Application Publication No. 2013/0095079. Incorporated herein by reference in its entirety.) In contrast, a 3D culture system (as described in Example 2) significantly preserved the CD34⁺ population. In addition, culture of HSPCs in the 3D alginate-poly-L-lysine matrix (3D ALG+PLL) culture showed a significantly larger CD34⁺ population than an alginate matrix without poly-L-lysine (3D ALG). More importantly, the cells from the 3D ALG+PLL culture system showed a much higher frequency of the HSPC population, which was comparable to or higher than uncultured HSPCs.

The 3D ALG+PLL system also minimized lineage differentiation of HSPCs after 14 days. As shown in Table 1 below, when cultured in the 2D Notch platform, significant expression of lineage markers by the cultured cells was observed after 14 days in culture. In contrast, the 3D culture platform (3D ALG+PLL) showed minimized lineage differentiation at day 14.

TABLE 1
Frequency of total CD34+ cells, CD34+ progenitors and HSCs/MPPs and
CD34 Lineage* cell in cell samples collected pre-culture (Day 0 (D0) and uncultured) and
cultured in the following formats for 14 days: 2D + Notch culture; 3D ALG and 3D
ALG + PLL static flasks; and 3D ALG + PLL roller bottles.

| | | 14 days post expansion | | | | |
|---|---|---|---|---|---|---|
| Percent | | | | | 3D ALG + | 3D ALG + |
| Frequency | D0 | Uncultured | 2D + Notch | 3D ALG | PLL Static | PLL Roller |
| Total CD34⁺ | 87.7 | 86.6 | 22.4 | 13.5 | 93.0 | 93.2 |
| HSC/MPP | 6.8 | 9.9 | 4.4 | 1.4 | 13.3 | 18.0 |
| Progenitors | 80.3 | 76.2 | 17.8 | 11.9 | 79.8 | 75.56 |
| Lineage* | 1.9 | 1.3 | 13.1 | 32.1 | 0.9 | 0.5 |
| Other | 10.5 | 10.9 | 64.1 | 54.1 | 6.2 | 6.4 |

HSC/MPP = Lin⁻ CD34⁺ CD38⁻ CD45RA⁻ CD133⁺ CD90^{+/-}
Progenitors = Lin⁻ CD34⁺ CD38⁺/Lin⁻ CD34⁺ CD38⁻ CD45RA⁺
Lineage+ = CD3⁺/CD14⁺/CD15⁺/CD16⁺/CD19⁺/CD20⁺

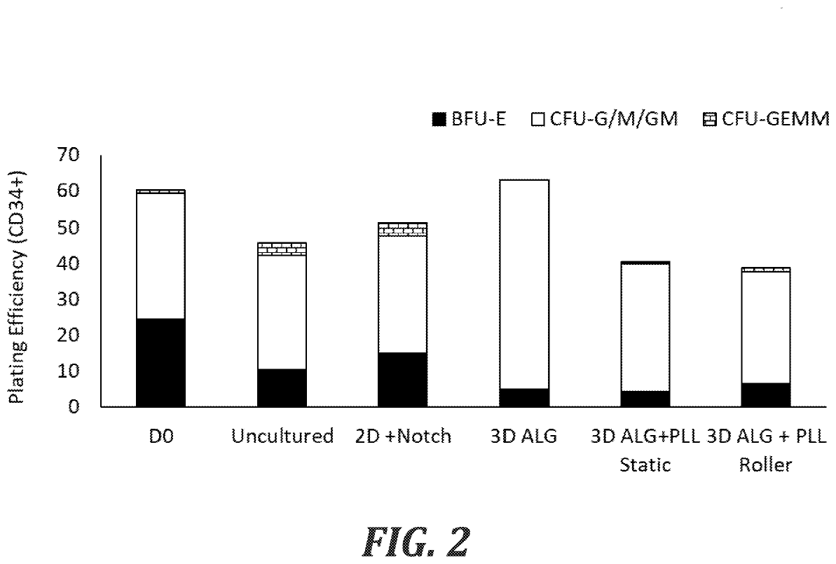

A CFC assay confirmed the retained colony-forming function of the harvested cells from 3D ALG+PLL culture. As shown in FIG. 2, cells harvested from a 3D ALG+PLL culture showed good and comparable functions to uncultured cells.

The in vivo engraftment capacity of cells from each group was also examined. Different number of cells from each group were injected into irradiated NSG mice: 1. Mock: 0 cells; 2. Uncultured: 100,000 TNC per mouse; 3. 2D Notch (DXI): 100,000, 500,000 and 7,300,000 TNC per mouse; 4. 3D ALG alone: 100,000 TNC per mouse; 5. 3D ALG/PLL static: 100,000 TNC per mouse; 6. 3D ALG/PLL Roller bottle: 100,000 TNC per mouse. Bone marrow and peripheral blood samples were analyzed at weeks 4, 8, 12, 16, 22 and 23 to determine the potential for engraftment and hematopoietic reconstitution in the recipient mice.

For the bone marrow analysis, as presented in Table 2, the highest total human engraftment was observed in the uncultured group at week 4. At the same time, and with the same number of infused cells, the 3D ALG+PLL groups also showed high total human engraftment (% human CD45⁺ (hCD45⁺). In contrast, with the same number of infused cells, the 3D ALG alone group and 2D Notch group showed a much more limited total engraftment at week 4. A higher total engraftment with increasing numbers of infused cells was observed from the 2D Notch group. In all positive engraftment groups (human chimerism >0.1%), a deeper analysis showed that a majority of repopulating cells have a myeloid phenotype (CD33⁺) and a HSC population can be found.

TABLE 2
The level of human engraftment (% hCD45), human myeloid population (CD45⁺
CD33⁺) and subpopulations of human HSPCs (HSC/MPP: Lin⁻ CD34⁺ CD38⁻ CD45RA⁻
CD133⁺ CD90^{+/-}; Progenitors: Lin⁻ CD34⁺ CD38⁺/Lin⁻ CD34⁺ CD38⁻ CD45RA⁺) in mouse
bone marrow at 4 weeks after primary transplantation are shown. Data from individual
mice for each group are averaged: N = 5 mice/group.
Week 4 Bone Marrow Analysis (Average Values/Group)

| | Condition | % | % | | % HSC/ | % | % |
|---|---|---|---|---|---|---|---|
| Group # | (Cell Dose) | hCD45* | CD34⁺ | Progenitors* | MPP* | CD33* | unknown |
| 1 | Mock (0) | 0.1 | 0.0 | 0.0 | 0.00 | 0.0 | 0.0 |
| 2 | Uncultured | 40.6 | 3.8 | 3.7 | 0.0 | 29.0 | 7.8 |
| | (1 × 10⁵) | | | | | | |
| 3 | 2D Notch | 0.3 | 0.1 | 0.1 | 0.00 | 0.2 | 0.0 |
| | (1 × 10⁵) | | | | | | |
| 4 | 2D Notch | 0.5 | 0.1 | 0.1 | 0.00 | 0.3 | 0.1 |
| | (5 × 10⁵) | | | | | | |
| 5 | 2D Notch | 12.1 | 1.5 | 1.5 | 0.00 | 8.8 | 1.9 |
| | (7.3 × 10⁶) | | | | | | |
| 6 | 3D ALG | 0.7 | 0.1 | 0.1 | 0.00 | 0.4 | 0.2 |
| | (1 × 10⁵) | | | | | | |
| 7 | 3D ALG + | 24.2 | 1.5 | 1.4 | 0.1 | 14.7 | 8.0 |
| | PLL Static | | | | | | |
| | (1 × 10⁵) | | | | | | |
| 8 | 3D ALG + | 16.8 | 2.8 | 2.8 | 0.0 | 9.7 | 4.4 |
| | PLL Roller | | | | | | |
| | (1 × 10⁵) | | | | | | |

*hCD45⁺ = Total human cell engraftment
*Progenitors = Lin⁻ CD34⁺ CD38⁺/Lin⁻ CD34⁺ CD38⁻ CD45RA⁺
*HSC/MPP = Lin⁻ CD34⁺ CD38⁻ CD45RA⁻ CD133⁺ CD90^{+/-}
*CD33⁺ = Myeloid

For the peripheral blood analysis, at weeks 4, 8, 12, 16 and 22, a similar observation to the bone marrow analysis at week 4 was found. As shown in Table 3, a higher portion of human chimerism was observed in the uncultured and 3D ALG+PLL groups. Among the human cells, different lineages were detected in the peripheral blood. At weeks 12 and 22, as presented in Tables 4 and 5 respectively, the 3D ALG+PLL groups and uncultured cells showed the highest human cell chimerism. Different from week 4, at week 12, a significant B cell population (CD20⁺ cell) was observed.

TABLE 3
The level of human engraftment (% hCD45) and human lineage
subpopulations in mouse blood 4 weeks after primary transplantation are shown. Data from
individual mice for each group were averaged: N = 5 mice/group.
Week 4 Peripheral Blood Analysis (Average Values/ Group)

| | Condition | | | | | | | | |
|---|---|---|---|---|---|---|---|---|---|
| Group | (Cell | | | | | | | | |
| # | Dose) | % hCD45⁺ | % CD4/8⁺ | % CD14/15⁺ | % CD20⁺ | % CD56⁺ | % CD7⁺ | % CD33⁺ | % Unknown+ |
| 1 | Mock (0) | 0.1 | 0.0 | 0.0 | 0.0 | 0.00 | 0.0 | 0.0 | 0.00 |
| 2 | Uncultured | 5.7 | 0.0 | 0.7 | 1.5 | 1.1 | 0.3 | 0.3 | 1.8 |
| | (1 × 10⁵) | | | | | | | | |
| 3 | 2D Notch | 0.1 | 0.0 | 0.0 | 0.00 | 0.00 | 0.00 | 0.0 | 0.00 |
| | (1 × 10⁵) | | | | | | | | |
| 4 | 2D Notch | 0.1 | 0.0 | 0.0 | 0.0 | 0.0 | 0.0 | 0.0 | 0.0 |
| | (5 × 10⁵) | | | | | | | | |
| 5 | 2D Notch | 0.8 | 0.0 | 0.1 | 0.1 | 0.1 | 0.2 | 0.1 | 0.2 |
| | (7.3 × 10⁶) | | | | | | | | |
| 6 | 3D ALG | 0.1 | 0.00 | 0.0 | 0.0 | 0.0 | 0.00 | 0.0 | 0.0 |
| | (1 × 10⁵) | | | | | | | | |
| 7 | 3D ALG + | 2.9 | 0.0 | 0.4 | 0.7 | 0.4 | 0.3 | 0.2 | 0.9 |
| | PLL Static | | | | | | | | |
| | (1 × 10⁵) | | | | | | | | |
| 8 | 3D ALG + | 2.7 | 0.0 | 0.3 | 0.6 | 0.3 | 0.3 | 0.2 | 0.8 |
| | PLL Roller | | | | | | | | |
| | (1 × 10⁵) | | | | | | | | |

hCD45⁺ = total human cell engraftment
CD4/8 = T Cells;
CD14/15 = Monocytes/Granulocytes;
CD20 = B Cells;
CD56 = NK Cells
CD33 = Myeloid

TABLE 4
The level of human engraftment (% hCD45) and human lineage subpopulations
in mouse blood 12 weeks after primary transplantation are shown. Data from individual
mice for each group were averaged: N = 5 mice/group.
Week 12 Peripheral Blood Analysis (Average Values/Group)

| Group | Condition | | | | | | | | | |
|---|---|---|---|---|---|---|---|---|---|---|
| # | (Cell Dose) | % hCD45⁺ | % CD34⁺ | % CD3⁺ | % CD14/15⁺ | % CD20⁺ | % CD56⁺ | % CD7⁺ | % CD33⁺ | % Unknown⁺ |
| 1 | Mock (0) | 0.0 | 0.0 | 0.0 | 0.0 | 0.0 | 0.0 | 0.0 | 0.0 | 0.0 |
| 2 | Uncultured | 19.7 | 0.1 | 0.2 | 0.6 | 16.9 | 0.1 | 0.1 | 0.5 | 0.9 |
| | (1 × 10⁵) | | | | | | | | | |
| 3 | 2D Notch | 0.6 | 0.3 | 0.0 | 0.2 | 0.1 | 0.0 | 0.0 | 0.0 | 0.0 |
| | (1 × 10⁵) | | | | | | | | | |
| 4 | 2D Notch | 0.2 | 0.0 | 0.0 | 0.1 | 0.1 | 0.0 | 0.0 | 0.0 | 0.0 |
| | (5 × 10⁵) | | | | | | | | | |
| 5 | 2D Notch | 3.9 | 0.1 | 0.1 | 0.2 | 2.3 | 0.1 | 0.1 | 0.0 | 0.2 |
| | (7.3 × 10⁶) | | | | | | | | | |
| 6 | 3D ALG | 0.4 | 0.1 | 0.0 | 0.1 | 0.1 | 0.0 | 0.0 | 0.0 | 0.0 |
| | (1 × 10⁵) | | | | | | | | | |
| 7 | 3D ALG + | 21.5 | 0.3 | 0.3 | 0.7 | 18.1 | 0.1 | 0.2 | 0.5 | 0.9 |
| | PLL Static | | | | | | | | | |
| | (1 × 10⁵) | | | | | | | | | |
| 8 | 3D ALG + | 22.0 | 0.2 | 0.4 | 0.7 | 18.2 | 0.1 | 0.2 | 0.6 | 1.2 |
| | PLL Roller | | | | | | | | | |
| | (1 × 10⁵) | | | | | | | | | |

hCD45⁺ = total human cell engraftment
CD4/8 = T Cells;
CD14/15 = Monocytes/Granulocytes;
CD20 = B Cells;
CD56 = NK Cells
CD33 = Myeloid

Long-term engraftment of the uncultured and expanded cells from all groups was assessed at 22 weeks. As shown in Table 5, the 3D ALG+PLL static and roller bottle cultured cells demonstrated long-term and multi-lineage engraftment at week 22 at levels comparable to the uncultured CD34⁺ cells. The 2D Notch expanded cells infused at the highest dose also demonstrated long-term multi-lineage engraftment but at lower levels. CD3⁺ T cell development was significantly increased by week 22 resulting in loss of some mice in groups with the highest engraftment levels (uncultured cells, 3D ALG+PLL static and roller bottle and highest dose of 2D Notch expanded cells) due to graft-vs-host disease (GVHD). No long-term engraftment was observed in remaining groups (3D ALG and lower doses of 2D Notch expanded cells).

TABLE 5
The level of human engraftment (% hCD45) and human lineage subpopulations
in mouse blood 22 weeks after primary transplantation are shown. Data from individual
mice for each group are averaged: N = 5 mice for all groups except uncultured cells (N =
4 mice), 3D ALG + PLL roller bottle (N = 3 mice).
Week 22 Peripheral Blood Analysis (Average Values/Group)

| Group | Condition | | | | | | | | |
|---|---|---|---|---|---|---|---|---|---|
| # | (Cell Dose) | % hCD45⁺ | % CD3⁺ | % CD14/15⁺ | % CD20⁺ | % CD56⁺ | % CD7⁺ | % CD33⁺ | % Unknown⁺ |
| 1 | Mock (0) | 0.0 | 0.0 | 0.0 | 0.0 | 0.0 | 0.0 | 0.0 | 0.0 |
| 2 | Uncultured | 51.6 | 24.7 | 1.0 | 23.0 | 0.6 | 0.5 | 0.3 | 0.9 |
| | (1 × 10⁵) | | | | | | | | |
| 3 | 2D Notch | 0.1 | 0.1 | 0.0 | 0.0 | 0.0 | 0.0 | 0.0 | 0.0 |
| | (1 × 10⁵) | | | | | | | | |
| 4 | 2D Notch | 0.4 | 0.2 | 0.0 | 0.3 | 0.0 | 0.0 | 0.0 | 0.0 |
| | (5 × 10⁵) | | | | | | | | |
| 5 | 2D Notch | 30.3 | 25.2 | 0.3 | 3.5 | 0.5 | 0.3 | 0.2 | 0.2 |
| | (7.3 × 10⁶) | | | | | | | | |
| 6 | 3D ALG | 1.5 | 0.0 | 0.0 | 1.3 | 0.0 | 0.0 | 0.0 | 0.1 |
| | (1 × 10⁵) | | | | | | | | |
| 7 | 3D ALG + | 38.4 | 17.2 | 1.1 | 17.0 | 0.6 | 0.7 | 0.4 | 0.9 |
| | PLL Static | | | | | | | | |
| | (1 × 10⁵) | | | | | | | | |
| 8 | 3D ALG + | 32.3 | 13.3 | 0.8 | 16.0 | 0.5 | 0.5 | 0.3 | 0.7 |
| | PLL Roller | | | | | | | | |
| | (1 × 10⁵) | | | | | | | | |

hCD45⁺ = total human cell engraftment
CD4/8 = T Cells;
CD14/15 = Monocytes/Granulocytes;
CD20 = B Cells;
CD56 = NK Cells
CD33 = Myeloid

Mice were sacrificed at week 23 and final immunophenotyping analysis was performed on bone marrow samples. As presented in Table 6, only groups which showed engraftment in peripheral blood at week 22 showed engraftment in the bone marrow as well. The highest total human engraftment at week 23 was observed in the 3D ALG+PLL static group with 59% human chimerism. Mice infused with uncultured CD34⁺ cells also showed high levels of engraftment (46% hCD45⁺), followed by mice infused with cells expanded for 14 days on 3D ALG+PLL (Roller Bottle; 39% hCD45⁺), 2D Notch (33% hCD45⁺) and 3D ALG (3.7% hCD45⁺). In all the above groups, a distinct population of CD34⁺ cells indicative of HSPCs, was detected.

TABLE 6
The level of human engraftment (% hCD45), and subpopulations of
human HSPCs (CD34⁺) and committed T cells, B cells, NK cells, monocytes and
neutrophils (Lineage⁺) in mouse bone marrow at 23 weeks after primary transplantation
are shown. Data from individual mice for each group are averaged: N = 5 mice for all
groups except Uncultured cells (N = 4 mice), 3D ALG + PLL roller bottle (N = 3 mice).
Week 23 Bone Marrow Analysis (Average Values/Group)

| | Condition | | | | | |
|---|---|---|---|---|---|---|
| Group # | (Cell Dose) | % hCD45* | % CD34⁺ | % Lineage⁺* | % CD7 | % unknown |
| 1 | Mock (0) | 0.0 | 0.0 | 0.0 | 0.0 | 0.0 |
| 2 | Uncultured | 46.3 | 7.2 | 39.3 | 0.3 | 4.3 |
| | (1 × 10⁵) | | | | | |
| 3 | 2D Notch | 0.1 | 0.0 | 0.1 | 0.0 | 0.0 |
| | (1 × 10⁵) | | | | | |
| 4 | 2D Notch | 0.3 | 0.1 | 0.1 | 0.0 | 0.1 |
| | (5 × 10⁵) | | | | | |
| 5 | 2D Notch | 32.8 | 2.6 | 36.3 | 0.2 | 2.8 |
| | (7.3 × 10⁶) | | | | | |
| 6 | 3D ALG | 3.7 | 0.4 | 2.5 | 0.0 | 0.8 |
| | (1 × 10⁵) | | | | | |
| 7 | 3D ALG + | 59.1 | 6.3 | 51.5 | 0.4 | 7.0 |
| | PLL Static | | | | | |
| | (1 × 10⁵) | | | | | |
| 8 | 3D ALG + | 39.1 | 6.2 | 31.6 | 0.3 | 5.0 |
| | PLL Roller | | | | | |
| | (1 × 10⁵) | | | | | |

*hCD45⁺ = Total human cell engraftment
*Lineage⁺ = CD3/CD4/CD8/CD20/CD14/CD15/CD56

In this evaluation, the 3D ALG+PLL culture supported modest expansion of cord blood derived CD34⁺ HSPCs and inhibited differentiation into lineage committed cells after ex vivo culture for 14 days while the expanded HSPCs retained their functional potency as evidenced by in vitro colony formation and in vivo long-term engraftment in NSG mice at levels comparable to uncultured enriched CD34⁺ HSPCs.

### Example 5: Using a 3D ALG+PLL Culture System to Expand Adult Peripheral Blood HSPCs

Adult HSPCs were prepared from healthy donors by first mobilizing CD34⁺ HSPCs to their peripheral blood by daily administration of G-CSF (filgastrim; 16 μg/kg BID; days 1-6) and/or plerixafor (240 μg/kg/day; days 4-6). Circulating CD34⁺ blood cell counts were analyzed daily and large volume leukapheresis was performed when CD34⁺ blood cell counts were >5 cells/μl. Following initial platelet wash, leukapheresis products were subjected to CD34⁺ HSPC cell isolation using standard techniques (including CliniMACS CD34⁺ positive selection).

Enriched adult CD34⁺ HSPCs were expanded using both the 3D ALG+PLL and the 2D Notch culture systems as described in Example 4 above. In addition, culture of the adult HSPCs in both platforms was also parallelly assessed in the CTS™ StemPro™ HSC Expansion Medium (StemPro).

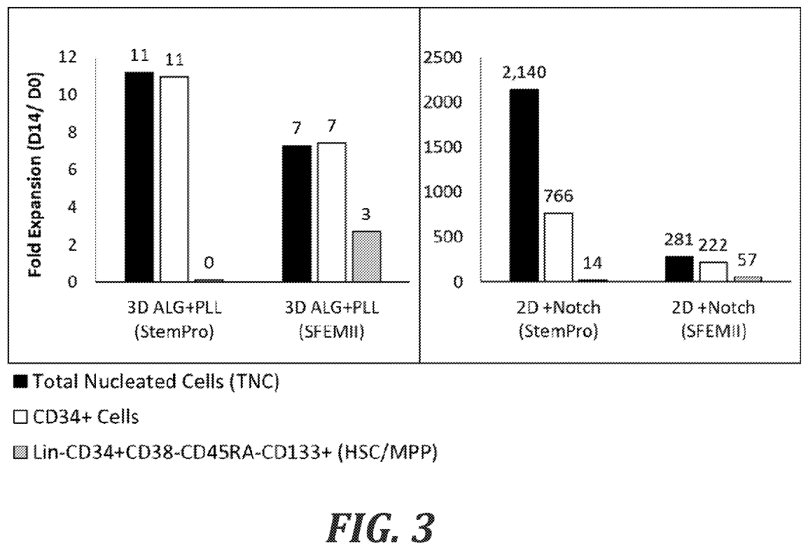

As shown in FIG. 3, following culture of enriched adult CD34⁺ HSPCs for 14 days, a significant expansion of total nucleated cells (TNC), CD34⁺ cells and HSCs/MPPs (Lin-CD34⁺ CD38⁻ CD45RA⁻ CD133⁺ CD90^{+/-} cells) was observed in the 2D culture system (using the Notch ligand DXI). This culture platform also supported terminal differentiation of the cultured HSPCs into Lineage⁺ cells as shown in Table 7.

In comparison, expansion of TNC and CD34⁺ cells was also observed in the 3D ALG+PLL system but at lower levels (FIG. 3). Similar to Example 4 above, the 3D ALG+PLL system also demonstrated inhibition of terminal differentiation of cultured HSPCs into Lineage⁺ cells (Table 7). Expansion of the HSC/MPP cell population in the 3D ALG+PLL system was only achieved with SFEM II media and not with StemPro culture media. Notably, SFEM II culture media was superior to StemPro culture media in supporting the expansion of HSC/MPP cell populations in both the 2D Notch and 3D ALG+PLL culture systems (FIG. 3). As shown in Table 7, the inhibition of terminal differentiation into Lineage⁺ cells was maintained during this 14-day ex vivo culture only with SFEM II media.

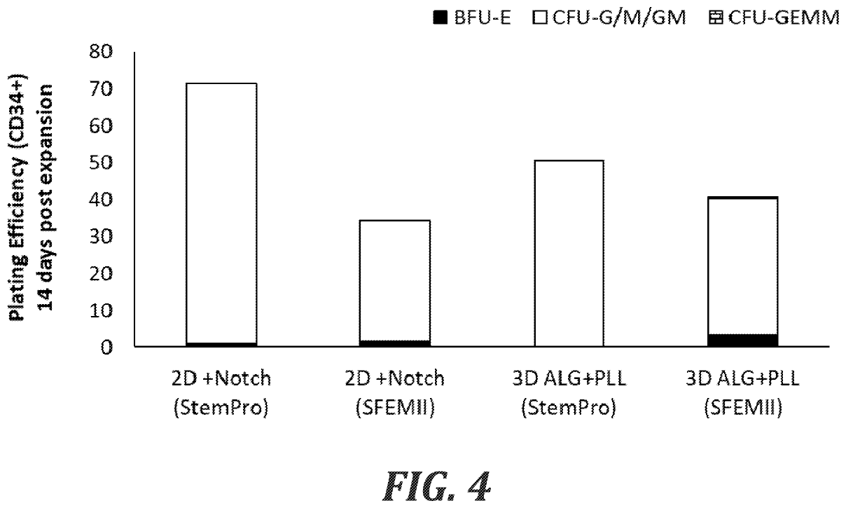

Functional potency of cells following culture in all four culture systems was evaluated by in vitro colony forming assays as described in Example 4. Only cells cultured in the 3D ALG+PLL culture system in SFEM II culture media were able to retain the potential to form all three colony morphologies (BFU-E, CFU-G/M/GM and CFU-GEMM) which is indicative of a multi-potent HSPC phenotype. Remaining culture systems showed biased colony morphologies toward CFU-G/M/GM morphologies indicating a committed progenitor phenotype (FIG. 4).

TABLE 7
Frequency of total CD34⁺ cells, multipotent HSCs/MPPs, lymphoid and myeloid
progenitors and CD34 Lineage⁺ cells in cell samples collected pre-culture (DO) and
cultured in the following formats for 14 days: 2D + Notch culture in SFEM II media;
2D + Notch culture in StemPro media; 3D ALG + PLL culture in SFEM II media; and 3D
ALG + PLL culture in StemPro media.

| | | 14 days post expansion | | | |
|---|---|---|---|---|---|
| Percent | | 2D + Notch | 2D + Notch | 3D ALG + PLL | 3D ALG + PLL |
| Frequency | D0 | (StemPro) | (SFEMII) | (StemPro) | (SFEMII) |
| HSC/MPP | 28.4 | 0.2 | 5.8 | 0.2 | 10.5 |
| Progenitors | 61.8 | 34.3 | 71.3 | 93.9 | 88.3 |
| Lineage* | 0.8 | 36.5 | 4.1 | 1.5 | 0.2 |
| Other | 6.8 | 27.9 | 17.9 | 2.9 | 0.4 |
| Total | 97.8 | 98.8 | 99.1 | 98.5 | 99.4 |

HSC/MPP = Lin⁻ CD34⁺ CD38⁻ CD45RA⁻ CD133⁺ CD90^{+/-}
Progenitors = Lin⁻ CD34⁺ CD38⁺/Lin− CD34⁺ CD38⁻ CD45RA⁺
Lineage⁺ = CD3⁺/CD14⁺/CD15⁺/CD16⁺/CD19⁺/CD20⁺

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications, including patents, patent application publications, and scientific literature, are cited herein, the disclosures of which are incorporated by reference in their entireties for all purposes.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of culturing hematopoietic stem and progenitor cells (HSPCs), comprising:
providing at least one HSPC population comprising hematopoietic stem cells (HSCs; Lin⁻ CD34⁺ CD38⁻ CD133⁺ CD45RA⁻ CD90⁺);
enriching the HSPC population for CD34⁺ or CD133⁺ HSPCs to prepare an enriched HSPC population that has been depleted of T cells and red blood cells;
encapsulating the enriched HSPCs in a zwitterionic hydrogel comprising alginate and poly-lysine to form encapsulated HSPCs; and
culturing the hydrogel encapsulated HSPCs in a culture medium comprising interleukin-3 (IL-3), interleukin-6 (IL-6), thrombopoietin (TPO), Flt3-Ligand (Flt3-L), and stem cell factor (SCF), for a sufficient time to produce a hydrogel expanded HSPC population, wherein the percentage of and/or the number of HSCs in the hydrogel expanded HSPC population is the same as or greater than the percentage of and/or number of HSCs in the enriched HSPCs.

2. A method of culturing hematopoietic stem and progenitor cells (HSPCs), comprising:
providing at least one enriched CD34⁺ or CD133⁺ HSPC population comprising hematopoietic stems cells (HSCs; Lin⁻ CD34⁺ CD38⁻ CD133⁺ CD45RA⁻ CD90⁺), less than 2% T cells and less than 2% red blood cells;
encapsulating the enriched HSPCs in a zwitterionic hydrogel comprising alginate and poly-lysine to form hydrogel encapsulated HSPCs; and
culturing the hydrogel encapsulated HSPCs in a culture medium comprising interleukin-3 (IL-3), interleukin-6 (IL-6), thrombopoietin (TPO), Flt3-Ligand (Flt3-L), and stem cell factor (SCF), for a sufficient time to produce a hydrogel expanded HSPC population, wherein the percentage and/or number of HSCs in the hydrogel expanded HSPC population is the same or greater than the percentage and/or number of HSCs in the enriched HSPCs.

3. The method of any one of the preceding claims, wherein the percentage of multi-potent progenitor cells (MPPs; Lin⁻ CD34⁺ CD38⁻ CD133⁺ CD45RA⁻ CD90⁻) in the expanded HSPC population is the same as or greater than the percentage and/or number of MPPs in the enriched HSPCs.

4. The method of any one of the preceding claims, wherein the enriched HSPC population is derived from umbilical cord blood, placental blood, and/or somatic stem cells.

5. The method of any one of the preceding claims, wherein the enriched HSPC population is derived from somatic stems cell of PBMCs.

6. The method of any one of the preceding claims, wherein the enriched HSPC population is derived from umbilical cord blood and/or placental blood.

7. The method of any one of the preceding claims, wherein the enriched HSPC population is derived from at least two different sources of umbilical cord blood and/or placental blood that have not been immunologically matched to each other, or to a recipient.

8. The method of any one of the preceding claims, wherein the culture medium does not comprise a serum supplement or serum supplement replacement.

9. The method of any one of the preceding claims, wherein the culture medium does not comprise fetal bovine serum, human serum albumin, or human platelet lysate.

10. The method of any one of the preceding claims, wherein the culture medium does not comprise exogenous IL-15, IL-7, IL-2, G-CSF, GM-CSF, LIF, or MIP-1a.

11. The method of any one of the preceding claims, wherein the culture medium does not comprise an aryl hydrocarbon receptor antagonist.

12. The method of any one of the preceding claims, wherein the culture medium and hydrogel do not comprise fibronectin and/or fragments thereof.

13. The method of any one of the preceding claims, wherein the culture medium and hydrogel do not comprise exogenous feeder cells.

14. The method of any one of the preceding claims, wherein the culture medium and hydrogel do not comprise a Notch ligand.

15. The method of any one of claims 1 to 13, wherein a Notch ligand is attached to the hydrogel.

16. The method of any one of the preceding claims, wherein the enriched HSPCs are not derived from somatic cells, embryonic stem cells, or induced pluripotent stem cells.

17. The method of any one of the preceding claims, wherein the percentage of HSCs in the hydrogel expanded HSPC population is at least two times greater than the percentage of HSCs in the enriched HSPC population and or the number of HSCs in the hydrogel expanded HSPC population is at least 10 times the number of HSCs in the enriched HSPC population.

18. The method of any one of the preceding claims, wherein the percentage of HSCs in the hydrogel expanded HSPC population at the end of the culturing step is at least two times greater than the percentage of HSCs in the encapsulated HSPC population at the beginning of the culturing step and/or the number of HSCs in the hydrogel expanded HSPC population at the end of the culturing step is at least 10 times the number of HSCs in the encapsulated HSPC population at the beginning of the culturing step.

19. The method of any one of the preceding claims, wherein the percentage of HSCs and/or MPPs in the hydrogel expanded HSPC population at the end of the culturing step is at least two times greater than the percentage of HSCs and/or MPPS in the encapsulated HSPC population at the beginning of the culturing step and/or the number of HSCs and/or MPPs in the hydrogel expanded HSPC population at the end of the culturing step is at least 5 times, at least 10 times, at least 20 times, at least 40 times, at least 50 times, or more than the number of HSCs and/or MPPs in the encapsulated HSPC population at the beginning of the culturing step.

20. The method of any one of the preceding claims, wherein the enriched HSPC population has been genetically modified.

21. The method of any one of the preceding claims, wherein the encapsulated HSPC population is genetically modified during culturing.

22. The method of any one of the preceding claims, wherein the hydrogel expanded HSPC population has been genetically modified to introduce a wild type version of a gene into the genome of at least some of the HSPCs.

23. The method of any one of the preceding claims, wherein the encapsulated HSPC population is cultured for about 2 to about 21 days.

24. The method of any one of the preceding claims, wherein the encapsulated HSPC population is cultured for about 7 to about 15 days.

25. The method of any one of the preceding claims, wherein the enriched HSPCs comprise about 25% to about 95%, 50% to about 95% HSPCs or about 75% to about 95% HSPCs.

26. The method of any one of the preceding claims, wherein at least some of the HSCs in the expanded HSCs are quiescent.

27. The method of any one of the preceding claims, wherein the poly-lysine is poly-L-lysine.

28. The method of any one of the preceding claims, wherein the hydrogel expanded HSPC population comprises at least about 5%, at least about 10% or at least about 15% HSCs, or at least about 5%, at least about 10% or at least about 15% HSCs and MPPs, or wherein the HSCs or the HSCs and MPPs in the hydrogel expanded HSPC population are expanded at least 5 fold, at least 10 fold, at least 20 fold, or at least 40 fold.

29. The method of any one of the preceding claims, comprising releasing the hydrogel expanded HSPCs from the hydrogel.

30. The method of any one of the preceding claims, further comprising differentiating the expanded HSPCs into HPCs, T cells, NK cells, macrophage, CD20⁺ B cells, CD14⁺ monocytes, and/or CD15⁺ neutrophils.

31. A composition comprising an enriched hematopoietic stem and progenitor cell (HSPC) population, comprising:
at least about 25% or at least about 50% HSPCs comprising hematopoietic stem cells (HSCs; Lin⁻ CD34⁺ CD38⁻ CD133⁺ CD45RA⁻ CD90⁺), less than 2% T cells, and less than 2% red blood cells; and
encapsulated in a zwitterionic hydrogel comprising alginate and poly-lysine.

32. The composition of claim 31, wherein the composition further comprises a culture medium comprising interleukin-3 (IL-3), interleukin-6 (IL-6), thrombopoietin (TPO), Flt3-Ligand (Flt3-L), and stem cell factor (SCF).

33. The composition of any one of claims 31 and 32, wherein the HSPCs are derived from umbilical cord blood, placental blood, and/or somatic stem cells.

34. The composition of any one of claims 31-33, wherein the HSPCs are derived from PBMCs.

35. The composition of any one of claims 31-34, wherein the HSPCs are derived from umbilical cord blood and/or placental blood.

36. The composition of any one of claims 31-35, wherein the HSPCs are derived from at least two different sources of umbilical cord blood and/or placental blood that have not been immunologically matched to each other, or to a recipient.

37. The composition of any one of claims 32-36, wherein the culture medium does not comprise a serum supplement or a serum supplement replacement.

38. The composition of claim 37, wherein the culture medium does not comprise fetal bovine serum, human serum albumin, or human platelet lysate.

39. The composition of any one of claims 32-38, wherein the culture medium does not comprise exogenous IL-15, IL-7, IL-2, G-CSF, GM-CSF, LIF, or MIP-1a.

40. The composition of any one of claims 32-39, wherein the culture medium does not comprise an aryl hydrocarbon receptor antagonist.

41. The composition of any one of claims 32-40, wherein the culture medium and hydrogel do not comprise fibronectin and/or fragments thereof.

42. The composition of any one of claims 32-41, wherein the culture medium and hydrogel do not comprise exogenous feeder cells.

43. The composition of any one of claims 32-42, wherein the culture medium and hydrogel do not comprise a Notch ligand.

44. The composition of any one of claims 32-42, wherein a Notch ligand is attached to the hydrogel.

45. The composition of any of claims 31-44, wherein the HSPCs are not derived from somatic cells, embryonic stem cells, and/or induced pluripotent stem cells.

46. The composition of any one of claims 31-45, wherein the HSPCs have been genetically modified.

47. The composition of any one of claims 31-46, wherein the HSPCs have been genetically modified to introduce a wild type version of a gene into the genome of at least some of the HSPCs.

48. The composition of any of one of claims 31-47, wherein the HSPCs comprise about 50% to about 95% HSPCs or about 75% to about 95% HSPCs.

49. The composition of any one of claims 31-48, wherein at least some of the HSCs in the HSPCs are quiescent.

50. The composition of any one of claims 31-49, wherein the poly-lysine is poly-L-lysine.

51. The composition of any one of claims 32-50, wherein the HSPCs comprise at least about 5%, at least about 10% or at least about 15% HSCs, or at least about 5%, at least about 10% or at least about 15% HSCs and MPPs.
